(19) United States
(12) Patent Application Publication
(10) Pub. No.: US 2019/0200915 A1
(43) Pub. Date: Jul. 4, 2019

(54) DIGITAL BIOMARKERS FOR COGNITION AND MOVEMENT DISEASES OR DISORDERS

(71) Applicant: Hoffmann-La Roche Inc., Little Falls, NJ (US)

(72) Inventors: Mike Baker, Basel (CH); Shibeshih Mitiku Belachew, Basel (CH); Christian Gossens, Basel (CH); Michael Lindemann, Basel (CH)

(21) Appl. No.: 16/299,869

(22) Filed: Mar. 12, 2019

Related U.S. Application Data
(63) Continuation of application No. PCT/EP2017/073173, filed on Sep. 14, 2017.

(30) Foreign Application Priority Data
Sep. 14, 2016 (EP) 16 188 847.4

(51) Int. Cl. A61B 5/00; A61B 5/11

(52) U.S. Cl. CPC A61B 5/4082; A61B 5/1125; A61B 5/4088; A61B 5/4848; A61B 5/4029; A61B 5/486; A61B 5/4839; A61B 5/4005

(57) ABSTRACT

A method for assessing a cognition and movement disease or disorder in a subject suspected to suffer therefrom. A cognition and/or fine motoric activity parameter is determined from a dataset of activity measurements obtained from the subject using a mobile device. The determined activity parameter is compared to a reference, and the cognition and movement disease or disorder is assessed. Also disclosed is a method for identifying whether a subject will benefit from a therapy for a cognition and movement disease or disorder. The steps just described are performed along with the step of identifying the subject as one who benefits from the therapy if the cognition and movement disease or disorder is assessed. Also disclosed is a mobile device comprising a processor, at least one sensor, a database and software which is tangibly embedded in said device and, when running on said device, carries out the disclosed methods.

### RELATED APPLICATIONS

This application is a continuation of PCT/EP2017/073173, filed Sep. 14, 2017, which claims priority to EP 16 188 847.4, filed Sep. 14, 2016, the entire disclosures of both of which are hereby incorporated herein by reference.

### BACKGROUND

This disclosure relates to the field of diagnostics. More specifically, it concerns a method for assessing a cognition and movement disease or disorder in a subject suspected to suffer therefrom comprising the steps of determining at least one cognition and/or fine motoric activity parameter from a dataset of cognition and/or fine motoric activity measurements obtained from said subject using a mobile device and comparing the determined at least one cognition and/or fine motoric activity parameter to a reference, whereby the cognition and movement disease or disorder will be assessed. This disclosure also relates to a method for identifying whether a subject will benefit from a therapy for a cognition and movement disease or disorder comprising the steps of the method of the aforementioned disclosure and the further step of identifying the subject as a subject that benefits from the therapy if the cognition and movement disease or disorder is assessed. The present disclosure contemplates a mobile device comprising a processor, at least one sensor and a database as well as software which is tangibly embedded in said device and, when running on said device, carries out the method of this disclosure, a system comprising a mobile device comprising at least one sensor and a remote device comprising a processor and a database as well as software which is tangibly embedded in said device and, when running on said device, carries out the method and the use of the mobile device or system according to this disclosure for assessing a cognition and movement disease or disorder in a subject.

Cognition and movement diseases and disorders are typically characterized by impaired cognitive and/or motoric functions. The diseases and disorders are less frequent but nevertheless typically accompanied by severe complications for the affected patients in daily life. Various cognition and movement disorders may result in life-threatening conditions and are finally mortal.

The diseases and disorders have in common that impaired function of the central nervous system, the peripheral nervous system and/or the muscular system results in cognition and movement disabilities. The movement disabilities may be primary disabilities due to direct impairments of muscle cells and function or may be secondary disabilities caused by impairments of muscle control by the peripheral and/or central nervous system central, in particular, the pyramidal, extrapyramidal, sensory or cerebellar system. The impairment may involve damage, degradation, intoxication or injury of nervous and/or muscular cells.

Typical cognition and movement diseases and disorders include but are not limited to multiple sclerosis (MS), neuromyelitis optica (NMO) and NMO spectrum disorders, stroke, a cerebellar disorder, cerebellar ataxia, spastic paraplegia, essential tremor, myasthenia and myasthenic syndromes or other forms of neuromuscular disorders, muscular dystrophy, myositis or other muscular disorders, a peripheral neuropathy, cerebral palsy, extrapyramidal syndromes, Parkinson's disease, Huntington's disease, Alzheimer's disease, other forms of dementia, leukodystrophies, autism spectrum disorders, attention-deficit disorders (ADD/ADHD), intellectual disabilities as defined by DSM-5, impairment of cognitive performances and reserve related to aging, a polyneuropathy, motor neuron diseases and amyotrophic lateral sclerosis (ALS).

Among the most commonly known and severe diseases and disorders there are MS, stroke, Alzheimer's disease, Parkinson's disease, Huntington's disease and ALS.

Multiple sclerosis (MS) is a severe neurodegenerative disease which at present cannot be cured. Affected by this disease are approximately 2 to 3 million individuals worldwide. It is the most common disease of the central nervous system (CNS) that causes prolonged and severe disability in young adults. There is evidence supporting the concept that a B- and T cell-mediated inflammatory process against self-molecules within the white matter of the brain and spinal cord causes the disease. However, its etiology is still not well understood. It has been found that myelin-reactive T cells are present in both MS patients and healthy individuals. Accordingly, the primary abnormality in MS may involve more likely an impaired regulatory mechanisms leading to an enhanced T cell activation status and less stringent activation requirements. The pathogenesis of MS includes activation of encephalitogenic, i.e., autoimmune myelin-specific T cells outside the CNS, followed by an opening of the blood-brain barrier, T cell and macrophage infiltration, microglia activation and demyelination. The latter causes irreversible neuronal damage (see, e.g., Aktas 2005, Neuron 46,421-432, Zamvil 2003, Neuron 38:685-688).

It was shown more recently that besides T cells, B lymphocytes (expressing CD20 molecule) may play a central role in MS and influence the underlying pathophysiology through at least four specific functions:
1. Antigen presentation: B cells can present self neuroantigens to T cells and activate them (Crawford A, et al. J Immunol 2006; 176(6):3498-506; Bar-Or A, et al. Ann Neurol 2010; 67(4):452-61)
2. Cytokine production: B cells in patients with MS produce abnormal proinflammatory cytokines, which can activate T cells and other immune cells (Bar-Or A, et al. Ann Neurol 2010; 67(4):452-61; Lisak R P, et al. J Neuroimmunol 2012; 246(1-2):85-95)
3. Autoantibody production: B cells produce autoantibodies that may cause tissue damage and activate macrophages and natural killer (NK) cells (Weber M S, et al. Biochim Biophys Acta 2011; 1812(2):239-45)
4. Follicle-like aggregate formation: B cells are present in ectopic lymphoid follicle-like aggregates, linked to microglia activation, local inflammation, and neuronal loss in the nearby cortex (Serafini B, et al. Brain Pathol 2004; 14(2):164-74; Magliozzi R, et al. Ann Neurol 2010; 68(4):477-93)

Although there is sound knowledge about the mechanisms responsible for the encephalitogenicity, far less is known regarding the control mechanisms for regulating harmful lymphocyte responses into and within the CNS in a subject.

MS diagnosis is based at present on clinical investigations by a medical practitioner. Such investigations involve testing of the capabilities of a patient for certain physical activities. Several tests have been developed and are routinely applied by medical practitioners. These tests aim at assessing walking, balance, and other motoric abilities. Examples of currently applied tests are the Expanded Disability Status Scale (EDSS, www.neurostatus.net) or Multiple Sclerosis Functional Composite (MSFC). These tests require the presence of a medical practitioner for evaluation and assessment purposes and are currently performed ambulant at doctors' offices or hospitals. Very recently, there have been some efforts in monitoring MS patients using smartphone devices in order to collect data of MS patients in a natural setting (Bove 2015, Neurol Neuroimmunol Neuroinflamm 2 (6):e162).

Further, diagnostic tools are used in MS diagnosis. Such tools include neuroimaging, analysis of cerebrospinal fluid and evoked potentials. Magnetic resonance imaging (MRI) of the brain and spinal cord can visualize demyelination (lesions or plaques). Contrast agents containing gadolinium can be administered intravenously to mark active plaques and, differentiate acute inflammation from the existence of older lesions which are not associated with symptoms at the moment of the evaluation. The analysis of cerebrospinal fluid obtained from a lumbar puncture can provide evidence of chronic inflammation of the central nervous system. The cerebrospinal fluid can be analyzed for oligoclonal immunoglobulin bands, which are an inflammation marker present in 75-85% of people with MS (Link 2006, J Neuroimmunol. 180 (1-2): 17-28). However, none of the aforementioned techniques is specific to MS. Therefore, ascertainment of diagnosis may require repetition of clinical and MRI investigations to demonstrate dissemination in space and in time of the disease which is a prerequisite to MS diagnosis.

There are several treatments approved by regulatory agencies for relapsing-remitting multiple sclerosis which shall modify the course of the disease. These treatments include interferon beta-1a, interferon beta-1b, glatiramer acetate, mitoxantrone, natalizumab, fingolimod, teriflunomide, dimethyl fumarate, alemtuzumab, and daclizumab. The interferons and glatiramer acetate are first-line treatments that reduce relapses by approximately 30% (see, e.g., Tsang 2011, Australian family physician 40 (12): 948-55). Natalizumab reduces the relapse rate more than the interferons, however, due to issues of adverse effects it is a second-line agent reserved for those who do not respond to other treatments or patients with severe disease (see, e.g., Tsang 2011, loc. cit.). Treatment of clinically isolated syndrome (CIS) with interferons decreases the chance of progressing to clinically definite MS (Compston 2008, Lancet 372(9648): 1502-17). Efficacy of interferons and glatiramer acetate in children has been estimated to be roughly equivalent to that of adults (Johnston 2012, Drugs 72 (9): 1195-211).

Recently, new monoclonal antibodies such as ocrelizumab, alemtuzumab and daclizumab have shown potential as therapeutics for MS. The anti-CD20 B-cell targeting monoclonal antibody ocrelizumab has shown beneficial effects in both relapsing and primary progressive forms of MS in one phase 2 and 3 phase III trials (NCT00676715, NCT01247324, NCT01412333, NCT01194570).

MS is a clinically heterogeneous inflammatory disease of the CNS. Therefore, diagnostic tools are needed that allow a reliable diagnosis and identification of the present disease status and can, thus, aid an accurate treatment, in particular, for those patients suffering from progressing forms of MS. Improvements in monitoring of disease progression are also highly desired.

Stroke may occur as an ischemic stroke where the blood support is impaired due to obstruction of blood vessels or as hemorrhagic stroke resulting from injury of vessels and bleeding.

Signs and symptoms of a stroke may include typically one-sided movement/motoric or sensory impairments, problems of walking, speaking, hearing, spinning vertigo or abnormalities of vision (Donnan 2008, Lancet. 371 (9624): 1612-23). Said signs and symptoms often appear immediately or soon after the stroke has occurred. If symptoms last less than one or two hours it is known as a transient ischemic attack. Hemorrhagic strokes may also be accompanied by severe headache. The symptoms of a stroke can be permanent. Long term comorbid complications may include pneumonia or loss of bladder control.

The early diagnosis and treatment of stroke is decisive for the outcome. Current stroke diagnosis requires imaging techniques such as magnetic resonance imaging (MRI) scanning, Doppler ultrasound, or angiography, as well as neurological examination by a medical practitioner (see, e.g., Harbison 1999, Lancet. 353 (9168): 1935; Kidwell 1998, Prehospital Emergency Care. 2 (4): 267-73; Nor 2005, Lancet Neurology. 4 (11): 727-34).

There are more than 10 million people affected by stroke every year. In the developed world, stroke management has meanwhile become rather efficient due to stroke units. However, these specialized centers are not present in less developed parts of the world of aside from urban regions. The early detection of the disorder has a major influence on the outcome of stroke in patients. Accordingly, there is a need for early detection of signs and symptoms of stroke even aside from the competent stroke units and hospitals. Beyond stroke detection there is also a crucial need for properly assessing mid- to long-term disability outcomes associated with acute stroke treatment intervention as well as spontaneous and rehabilitation program-related recovery.

Alzheimer's disease is a severe and mortal neurodegenerative disease accompanied by dementia and associated problems. In fact, Alzheimer's disease is responsible for 60 to 70% of all cases of dementia. An early symptom of the disease is a reduced short-term memory. Subsequent symptoms include social symptoms such as withdrawal from family and society, as well as physical symptoms such as loss of body functions (Burns 2009, The BMJ. 338: b158).

Diagnosis of Alzheimer's disease is based on imaging techniques such as CT, MRI, SPECT or PET. Moreover, neurological assessments are carried out by medical practitioners including tests for assessment of cognitive functions (Pasquier 1999, Journal of Neurology 246 (1):6-15). Typical tests include tests where people are instructed to copy drawings similar to the one shown in the picture, remember words, read, and subtract serial numbers. Usually, caregivers are required for the diagnosis since the Alzheimer's disease patient him/herself is unaware of his/her deficits. There is no efficient disease-modifying treatment or cure yet for Alzheimer's disease. However, for an efficient disease management a reliable and early diagnosis is helpful.

Alzheimer's disease affects about 50 million people worldwide and may be one of the most frequent neurodegenerative diseases in the elderly. Accordingly, there is a need for early detection of signs and symptoms for a proper management of the disease as well as a need for monitoring of disease progression.

Parkinson's disease is a neurodegenerative disease of the central nervous system that pivotally affects the motoric system. Typical symptoms are resting tremor, postural instability, shaking, rigidity, slowness of movement, and difficulties with walking. Dementia and depression and sensory, autonomous nervous system and sleeping problems may also occur at more severe stages of the disease. The motoric problems are caused by degeneration of neurons in the substantia nigra of the midbrain resulting in a significant alteration of dopaminergic neurotransmission. There is no cure for Parkinson's disease available yet.

Diagnosis of Parkinson's disease is based on neurological assessments together with imaging methods, such as CT, MRI, PET or SPECT scanning. Neurological criteria for the diagnosis of the disease include the assessment of bradykinesia, rigidity, resting tremor and postural instability (Jankovic 2008, Journal of Neurology, Neurosurgery, and Psychiatry. 79 (4): 368-376).

More than 50 million people are affected by Parkinson's disease. There is a need for an early and reliable diagnosis of this neurodegenerative disease as well as monitoring disease progression.

Huntington's disease is an inherited disorder that results in death of neurons in the central nervous system and, in particular, in the brain. The earliest symptoms are often subtle problems with mood or mental abilities. However, general impairment of coordination and an unsteady gait typically occurs afterwards (Dayalu 2015, Neurologic Clinics. 33 (1): 101-14) In its advanced stages, uncoordinated body movements become apparent and physical abilities gradually worsen until coordinated movement becomes difficult and the person is unable to speak. The cognitive capabilities are also impaired and may decline into dementia (Frank 2014, The Journal of the American Society for Experimental NeuroTherapeutics. 11 (1): 153-60). The specific symptoms may, however, individually vary. There is no cure for Huntington's disease available yet.

Since Huntington's disease is inherited in a dominant autosomal manner, genome testing for CAG repeats in the huntingtin (HTT) alleles is recommended for individuals being genetically at risk, i.e., patients with a corresponding family history of the disease. Moreover, diagnosis of the disease involves DNA analysis but also imaging methods such as CT, MRI, PET or SPECT scanning, in order to determine cerebral atrophy as well as neurological assessment by a medical practitioner. In particular, the neurological assessments can be carried out according to the criteria for the unified Huntington's diseases rating scale system (Rao 2009, Gait Posture. 29 (3): 433-6).

Huntington's disease is less frequent than Alzheimer's disease and Parkinson's disease. However, it is still a cognition and movement disease or disorder affecting a significant proportion of people with severe and life-threatening complications. There is a need for an early and reliable diagnosis of this neurodegenerative disease as well as monitoring disease progression.

ALS is a neurodegenerative disease that involves cell death of the lower and upper motor neurons that control voluntary muscle contraction (Zarei 2015, Surgical Neurology International. 6: 171). ALS is characterized by stiff muscles, muscle twitching, amyotrophy, and gradually worsening weakness due to muscles decreasing in size resulting in difficulties in walking, speaking, swallowing, and breathing. Respiratory failure is usually the cause of death in patients suffering from ALS. There is no cure yet available for this mortal disease.

The diagnosis of ALS is difficult and requires ruling out other possible causes of symptoms and signs such as muscle weakness, muscle atrophy, impaired swallowing or breathing, cramping, or stiffness of affected muscles, and/or slurred and nasal speech. Besides neurological assessment by medical practitioners, the diagnosis typically involves EMG, measuring nerve conductive velocity or MRI. Laboratory tests including muscle biopsy are also available.

Nevertheless, there is a need for an early and reliable diagnosis of this neurodegenerative disease as well as monitoring of disease progression.

The aforementioned cognition and movement diseases and disorders are prominent examples which shall illustrate the need for an early and reliable diagnosis of the disease or disorder conditions, in particular, in daily life situations as well as for a monitoring of the disease condition and/or progression. However, such a reliable and efficient diagnosis currently requires the presence of a medical practitioner for neurological assessments or application of expensive and time consuming imaging methods in, e.g., hospitals. These drawbacks apply mutatis mutandis for the other cognition and movement diseases and disorders. Therefore, there is a need for less expensive, reliable and effective diagnostic tools and measures which can be carried out in a simple manner during daily life situations by the affected patients.

### SUMMARY

The technical problem underlying this disclosure may be seen in the provision of means and methods complying with the aforementioned needs. The technical problem is addressed by the embodiments described herein below.

This disclosure relates to a method for assessing a cognition and movement disease or disorder in a subject suspected to suffer therefrom comprising the steps of:
a) determining at least one cognition or fine motoric activity parameter from a dataset of cognition or fine motoric activity measurements obtained from said subject using a mobile device; and
b) comparing the determined at least one cognition or fine motoric activity parameter to a reference, whereby the cognition and movement disease or disorder will be assessed.

Typically, the method further comprises the step of (c) assessing the cognition and movement disease or disorder in a subject based on the comparison carried out in step (b).

In some embodiments, the method may also comprise prior to step (a) the step of obtaining from the subject using a mobile device a dataset of activity measurements during predetermined activity performed by the subject. However, typically the method is an ex vivo method carried out on an existing dataset of cognition or fine motoric activity measurements of a subject which does not require any physical interaction with the said subject.

The method as referred to in accordance with this disclosure includes a method which essentially consists of the aforementioned steps or a method which may include additional steps.

The method may be carried out on the mobile device by the subject once the dataset of activity measurements has been acquired. Thus, the mobile device acquiring the dataset and the device evaluating the dataset may be physically identical, i.e., the same device. Such a mobile device shall have a data acquisition unit which typically comprises means for data acquisition, i.e., means which detect or measure either quantitatively or qualitatively physical and/or chemical parameters and transform them into electronic signals transmitted to the evaluation unit in the mobile device used for carrying out the method according to this disclosure. The data acquisition unit comprises means for data acquisition, i.e., means which detect or measure either quantitatively or qualitatively physical and/or chemical parameters and transform them into electronic signals transmitted to the device being remote from the mobile device and used for carrying out the method according to this disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors and the like. The evaluation unit typically comprises a processor and a database as well as software which is tangibly embedded in said device and, when running on said device, carries out the method of this disclosure. More typically, such a mobile device may also comprise a user interface, such as a screen, which allows for providing the result of the analysis carried out by the evaluation unit to a user.

Alternatively, the method of this disclosure may be carried out on a device being remote with respect to the mobile device that has been used to acquire the said dataset. In this case, the mobile device shall merely comprise means for data acquisition, i.e., means which detect or measure either quantitatively or qualitatively physical and/or chemical parameters and transform them into electronic signals transmitted to the device being remote from the mobile device and used for carrying out the method according to this disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors and the like. Thus, the mobile device and the device used for carrying out the disclosed method may be physically different devices. In this case, the mobile device may correspond with the device used for carrying out the method of the present disclosure by any means for data transmission. Such data transmission may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Accordingly, for carrying out the method of this disclosure, the only requirement is the presence of a dataset of activity measurements obtained from a subject using a mobile device. The said dataset may also be transmitted or stored from the acquiring mobile device on a permanent or temporary memory device which subsequently can be used to transfer the data to the device used for carrying out the method of the present disclosure. The remote device which carries out the method of this disclosure in this setup typically comprises a processor and a database as well as software which is tangibly embedded in said device and, when running on said device, carries out the disclosed method. More typically, the said device may also comprise a user interface, such as a screen, which allows for providing the result of the analysis carried out by the evaluation unit to a user. Thus, the mobile device and the remote device in this setup form a system for carrying out the method of this disclosure.

The term “assessing” as used herein refers to assessing whether a subject suffers from the cognition and movement disease or disorder, or not, or whether a disease or disorder as referred to herein or individual symptoms thereof worsen or improve over time or dependent on a certain stimulation, or not. Accordingly, assessing as used herein includes identifying progression of the said cognition and movement disease or disorder or one or more symptoms accompanying it, identifying improvement of the said cognition and movement disease or disorder or one or more symptoms accompanying it, monitoring the said cognition and movement disease or disorder or one or more symptoms accompanying it, determining efficacy of a therapy of the said cognition and movement disease or disorder or one or more symptoms accompanying it, and/or diagnosing the said cognition and movement disease or disorder or one or more symptoms accompanying it. As will be understood by those skilled in the art, such an assessment, although preferred to be, may usually not be correct for 100% of the investigated subjects. The term, however, requires that a statistically significant portion of subjects can be correctly assessed and, thus, identified as suffering from the cognition and movement disease or disorder. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05. Thus, the method of this disclosure, typically, aids the assessment of cognition and movement diseases or disorders by providing a means for evaluating a dataset of activity measurements.

The term “the cognition and movement disease or disorder” as used herein relates to diseases that are accompanied by impaired cognition and/or movement disabilities. Typically, these diseases or disorders are caused by impaired function of the central nervous system, the peripheral nervous system or the muscular system. The impairment may involve damage or injury of nervous and/or muscular cells such as damages caused by neurodegenerative diseases such as multiple sclerosis, Alzheimer's disease, Chorea Huntington, Parkinson's disease or others. Typically, the cognition and movement disorder is a disease or disorder of the central and/or peripheral nervous system affecting the pyramidal, extrapyramidal, sensory or cerebellar system, or a neuromuscular disease or is a muscular disease or disorder. More typically, the said disease or disorder is selected from the group consisting of: multiple sclerosis (MS), neuromyelitis optica (NMO) and NMO spectrum disorders, stroke, a cerebellar disorder, cerebellar ataxia, spastic paraplegia, essential tremor, myasthenia and myasthenic syndromes or other forms of neuromuscular disorders, muscular dystrophy, myositis or other muscular disorders, a peripheral neuropathy, cerebral palsy, extrapyramidal syndromes, Parkinson's disease, Huntington's disease, Alzheimer's disease, other forms of dementia, leukodystrophies, autism spectrum disorders, attention-deficit disorders (ADD/ADHD), intellectual disabilities as defined by DSM-5, impairment of cognitive performances and reserve related to aging, Parkinson's disease, Huntington's disease, a polyneuropathy, motor neuron diseases and amyotrophic lateral sclerosis (ALS).

Multiple sclerosis (MS) is a typical cognition and movement disease or disorder according to this disclosure. There are four standardized subtype definitions of MS which are also encompassed by the term as used in accordance with this disclosure: relapsing-remitting, secondary progressive, primary progressive and progressive relapsing. The term relapsing forms of MS is also used and encompasses relapsing-remitting and secondary progressive MS with superimposed relapses. The relapsing-remitting subtype is characterized by unpredictable relapses followed by periods of months to years of remission with no new signs of clinical disease activity. Deficits suffered during attacks (active status) may either resolve or leave sequelae. This describes the initial course of 85 to 90% of subjects suffering from MS. Secondary progressive MS describes those with initial relapsing-remitting MS, who then begin to have progressive neurological decline between acute attacks without any definite periods of remission. Occasional relapses and minor remissions may appear. The median time between disease onset and conversion from relapsing remitting to secondary progressive MS is about 19 years. The primary progressive subtype describes about 10 to 15% of subjects who never have remission after their initial MS symptoms. It is characterized by progression of disability from onset, with no, or only occasional and minor, remissions and improvements. The age of onset for the primary progressive subtype is later than other subtypes. Progressive relapsing MS describes those subjects who, from onset, have a steady neurological decline but also suffer clear superimposed attacks. It is now accepted that this latter progressive relapsing phenotype is a variant of primary progressive MS (PPMS) and diagnosis of PPMS according to McDonald 2010 criteria includes the progressive relapsing variant.

Symptoms associated with MS include changes in sensation (hypoesthesia and par-aesthesia), muscle weakness, muscle spasms, difficulty in moving, difficulties with co-ordination and balance (ataxia), problems in speech (dysarthria) or swallowing (dysphagia), visual problems (nystagmus, optic neuritis and reduced visual acuity, or diplopia), fatigue, acute or chronic pain, bladder, sexual and bowel difficulties. Cognitive impairment of varying degrees as well as emotional symptoms of depression or unstable mood are also frequent symptoms. The main clinical measure of disability progression and symptom severity is the Expanded Disability Status Scale (EDSS). Further symptoms of MS are well known in the art and are described in the standard text books of medicine and neurology, such as for instance Bradley W G, et al. Neurology in Clinical Practice (5th ed. 2008).

Progressing MS as used herein refers to a condition, where the disease and/or one or more of its symptoms get worse over time. Typically, the progression is accompanied by the appearance of active statuses. The said progression may occur in all subtypes of the disease. However, typically progressing MS shall be determined in accordance with this disclosure in subjects suffering from relapsing-remitting MS.

However, the method of this disclosure can be applied, in particular, in the context of:
identifying clinical disease activity (i.e., relapse occurrence),
disability progression,
primary progressive MS disease course, as defined by established consensus criteria such as but not exclusively the McDonald Criteria 2010 (Polman 2011, Ann Neurol 69:292-302), and/or the Lublin et al. criteria 2013 (Lublin 2014, Neurology 83: 278-286),
secondary progressive MS disease course, as defined by established consensus criteria such as but not exclusively the McDonald Criteria 2010 (Polman loc. cit.), and/or the Lublin et al. criteria 2013 (Lublin loc. cit.),
primary progressive MS, as defined by established consensus criteria such as but not exclusively the McDonald Criteria 2010 (Polman loc. cit.), and/or the Lublin et al. criteria 2013 (Lublin loc. cit.), and/or
secondary progressive MS, as defined by established consensus criteria such as but not exclusively the McDonald Criteria 2010 (Polman loc. cit.), and/or the Lublin et al. criteria 2013 (Lublin loc. cit.).

Moreover, it is suitable for risk assessments in MS patients and, in particular for:
Risk prediction models estimating probabilities of disease activity (i.e., relapse and/or new or enlarging lesions on T2 or FLAIR (Fluid Attenuating Inversion Recovery) weighted brain or spinal cord MRI, and/or gadolinium-enhancing lesions on brain or spinal cord MRI),
risk prediction models estimating probabilities of disability progression in patients with a diagnosis of multiple sclerosis (MS), as measured for instance but not exclusively by the Expanded Disability Status Scale neurostatus (EDSS), the Multiple Sclerosis Functional Composite (MSFC), and its components the Timed 25 foot walk test or the 9-hole peg test, and/or
risk prediction models estimating probabilities of emergence of secondary progressive MS disease course in relapsing-onset MS as defined by established consensus criteria such as but not exclusively the McDonald Criteria 2010 (Polman loc. cit.), and/or the Lublin et al. criteria 2013 (Lublin loc. cit.).
risk prediction models estimating probabilities of emergence of specific MRI signs of primary or secondary progressive MS disease course as defined for instance but not exclusively by the presence of slowly expanding lesions (SELs) on T2 or FLAIR weighted brain or spinal cord MRI, or signs of meningeal inflammation detected on FLAIR-weighted brain or spinal cord MRI after injection of gadolinium-based contrast agents.

Furthermore, the method can be applied in the context of:
Developing algorithmic solutions using for instance machine-learning and pattern recognition techniques to estimate probabilities of disease-modifying treatment (DMT) response or failure as evaluated by the risk of ongoing disease activity (i.e., relapse and/or new or enlarging lesions on T2 or FLAIR weighted brain or spinal cord MRI, and/or gadolinium-enhancing lesions on brain or spinal cord MRI) in patients with a diagnosis of multiple sclerosis (MS) treated with specific DMTs,
developing algorithmic solutions using for instance machine-learning and pattern recognition techniques to estimate probabilities of DMT response or failure as evaluated by the risk of ongoing disability progression in patients with a diagnosis of multiple sclerosis (MS) treated with specific DMTs, as measured for instance but not exclusively by the Expanded Disability Status Scale (EDSS), the Timed 25 foot walk test or the 9-hole peg test, and/or
developing algorithmic solutions using for instance machine-learning and pattern recognition techniques to estimate probabilities of DMT response or failure as evaluated by the risk of worsening in brain MRI measures of neural tissue damage and neurodegeneration such as but not exclusively the whole brain volume, brain parenchymal fraction, whole grey matter volume, cortical grey matter volume, volume of specific cortical areas, deep grey matter volume, thalamic volume, corpus callosum surface, white matter volume, third ventricle volume, total brain T2 lesion volume, total brain T1 lesion volume, total brain FLAIR lesion volume in patients with a diagnosis of multiple sclerosis (MS) treated with specific DMTs, algorithmic solutions using for instance machine-learning and pattern recognition techniques to estimate probabilities of emergence of secondary progressive MS disease course in relapsing-onset MS as defined by established consensus criteria such as but not exclusively the McDonald Criteria 2010 (Polman loc. cit.), and/or the Lublin et al. criteria 2013 (Lublin loc. cit.).

Neuromyelitis optica (NMO, previously known as Devic disease) and neuromyelitis optica spectrum disorders (NMOSD) are inflammatory disorders of the central nervous system characterized by severe, immune-mediated demyelination and axonal damage predominantly targeting optic nerves and spinal cord. Traditionally considered a variant of multiple sclerosis, NMO is now recognized as a distinct clinical entity based on unique immunologic features. The discovery of a disease-specific serum NMO-IgG antibody that selectively binds aquaporin-4 (AQP4) has led to increased understanding of a diverse spectrum of disorders. NMO and NMOSD are characterized by severe relapsing attacks of optic neuritis and transverse myelitis which, unlike the attacks in multiple sclerosis, commonly spare the brain in the early stages. The spectrum of NMO is traditionally restricted to the optic nerves and the spinal cord. The method of the present disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

A stroke as referred to herein refers to an impairment of the blood flow in the central nervous system, in particular in the brain. Stroke may be ischemic stroke caused by obstruction of a blood vessels and consequent lack of blood flow into a brain tissue area or may be hemorrhagic stroke caused by brain injury and subsequent bleeding. Symptoms of stroke depend on the affected brain area and typically may encompass one or more of the following ones: one-sided inability to move or to feel, problems of understanding or speaking, spinning, or partial loss of vision. A hemorrhagic stroke may also encompass severe headache. In any event, for the treatment of stroke the time period between the event and the treatment is crucial, in particular, in order to avoid long-term effects on cognition or other central nervous system functions. In some cases the symptoms of stroke may be rather mild and may not be easy to diagnose without suitable test equipment. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

A cerebellar disease according to this disclosure encompasses any disease which affects the function of the cerebellum. The cerebellum is involved in motor control and learning. Animals and humans with cerebellar dysfunction show, above all, problems with motor control, on the same side of the body as the damaged part of the cerebellum. They continue to be able to generate motor activity, but it loses precision, producing erratic, uncoordinated, or incorrectly timed movements. Typical manifestations of motoric problems arising from the cerebellum include hypotonia, dysmetria, dysarthria, dysdiadochokinesia, intentional tremor or gait impairments. Typically, the disorders causing the aforementioned disabilities are also called cerebellar ataxia. Other diseases affecting the cerebellum include degenerative disease such as olivopontocerebellar atrophy, Machado-Joseph disease, ataxia telangiectasia, Friedreich's ataxia, Ramsay Hunt syndrome type I, paraneoplastic cerebellar degeneration or prion diseases, or may be congenital malformation or underdevelopment (hypoplasia) of the cerebellar vermis, such as Dandy-Walker syndrome or Joubert syndrome. In addition, cerebellar atrophy may also cause cerebellar diseases and may occur in Huntington's disease, multiple sclerosis, essential tremor, progressive myoclonus epilepsy, Niemann-Pick disease, as a result of exposure to toxins including heavy metals or pharmaceutical or recreational drugs or from an acute deficiency of vitamin B1 (thiamine) as seen in beriberi and in Wernicke-Korsakoff syndrome or from vitamin E deficiency. The method of the present disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Spastic paraplegia as used herein refers to a group of inherited diseases accompanied by progressive stiffness and spasticity in the lower limbs. The diseases may also affect the optic nerve, the retina, cause cataracts, ataxia, epilepsy, cognitive impairment, peripheral neuropathy, and deafness. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Essential tremor as used herein refers to a movement disorder involving tremors of the arms, hands, and fingers. Sometimes, other body parts and the voice may also be affected by tremors. Essential tremor is typically an action tremor (i.e., it occurs if the affected muscle shall be used) or a postural tremor (it is present with sustained muscular tone). The method of the present disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Myasthenia as used herein refers to a neuromuscular disease also called myasthenia gravis characterized by frequently occurring muscle weakness and fatigue. The muscle weakness becomes more pronounced during exercise and less pronounced at periods of rest. It is caused by circulating autoantibodies that block nicotinic acetylcholine receptors. These antibodies prevent motor neurons from transmitting signals towards the muscles. There are other forms of myasthenia related neuromuscular disease such as ocular myasthenia or Lambert-Eaton myasthenia syndrome. Said other forms of neuromuscular disorders are also envisaged by this disclosure as cognition and movement disorders and diseases. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Muscular dystrophy as referred to in accordance with this disclosure relates to a weakening of muscle caused by defects or death of muscle cells and tissue. Typically, muscle proteins such as dystrophin may become greatly reduced in muscle dystrophy. Muscular dystrophy as referred to herein encompasses but is not limited to Becker muscular dystrophy, congenital muscular dystrophy, Duchenne muscular dystrophy, distal muscular dystrophy, Emery-Dreifuss muscular dystrophy, facioscapulohumeral muscular dystrophy, limb-girdle muscular dystrophy, and myotonic muscular dystrophy. Moreover, also encompassed in accordance with the present disclosure are forms of myositis or other muscular disorders.

Peripheral neuropathy as used herein refers to a disease wherein the proper function of peripheral nerves is impaired. Typically, the nerves envisaged in accordance with this disclosure are those required for movements or sensation. These neuropathies are also called motor neuropathy or sensory neuropathy. Motor neuropathy may cause impaired balance and coordination or, most typically, muscle weakness. Sensory neuropathy may cause numbness to touch and vibration, or reduced position sense causing poorer coordination and balance, but also reduced sensitivity to temperature change and pain, spontaneous tingling or burning pain, or skin allodynia. Neuropathies may also further be classified as mononeuropathy wherein essentially a single nerve is affected and polyneuropathies affecting various nerves in different parts of the body. Different causes for neuropathies have been described involving severe disease, such as diabetes, immune disease, infections, physical injuries, chemotherapy, radiation therapy, cancer, alcoholism, Beriberi, hypothyroidism, porphyria, vitamin B12 deficiencies, or excessive vitamin B6. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Polyneuropathy is understood as damage or disorder affecting peripheral nerves in roughly the same areas on both sides of the body. Polyneuropathies may be classified in different ways, such as by cause, by speed of progression, by the parts of the body involved or by part of the nerve cell (axon, myelin sheath, or cell body) that is mainly affected. Polyneuropathy can further be classified as acute polyneuropathy, for example caused by infections, autoimmune reactions, toxins, certain drugs or cancer, and chronic polyneuropathy, for example caused by diabetes mellitus, excessive alcohol consumption or degeneration of nerves. Symptoms of polyneuropathy include weakness, numbness, or burning pain which usually begin in the hands and feet and may progress to the arms, legs and sometimes to other parts of the body (Burns 2011, Neurology 76.7 Supplement 2: S6-S13). A number of different disorders are known to cause polyneuropathy, for example diabetes and some types of Guillain-Barré syndrome. Diagnosis of polyneuropathy is commonly based on physical examinations and further clinical tests including, for example, electromyography, nerve conduction studies, muscle biopsy or certain antibody tests. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Cerebral palsy (CP) is a group of permanent movement disorders. CP usually appears in early childhood and is caused by abnormal development or damage to the parts of the brain that control movement, balance, and posture. Symptoms include poor coordination, stiff muscles, weak muscles, tremors, seizures, a decreased ability to think or reason, problems with sensation, vision, hearing, swallowing, and speaking. According to the Centers for Disease Control and Prevention (CDC), CP is the most common movement disorder in children and has a prevalence of about 2.11 per 1,000 live births. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Extrapyramidal syndromes (EPS) are considered to be drug-induced movement disorders. The term “Extrapyramidal symptoms” is derived from the fact that they are symptoms of disorders in the extrapyramidal system that normally regulates posture and skeletal muscle tone. Symptoms may be acute or tardive and include dystonia (continuous spasms and muscle contractions), akathisia (motor restlessness), parkinsonism (characteristic symptoms such as rigidity), bradykinesia (slowness of movement), tremor, and tardive dyskinesia (irregular, jerky movements). Extrapyramidal syndromes are most commonly caused by antipsychotic or antidepressant drugs such as haloperidol, fluphenazine, duloxetine, sertraline, escitalopram, fluoxetine, and bupropion. The method of the present disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Alzheimer's disease (AD) is a chronic neurodegenerative disease. The disease course of AD can be divided into four stages, with a progressive pattern of cognitive and functional impairment: Pre-dementia, Early stage, Moderate stage, and Advanced stage.

The pre-dementia stage of the disease has also been termed mild cognitive impairment (MCI) and includes early symptoms of AD such as short-term memory loss and difficulties in planning or solving problems (Waldemar 2007, European Journal of Neurology 14.1: e1-e26; Bäckman 2004, Journal of internal medicine 256.3: 195-204). In the early stage of AD, symptoms such as problems with language, executive functions, perception (agnosia) and execution of movements (apraxia) become apparent. As the disease progresses, behavioral and neuropsychiatric changes become more prevalent. The moderate phase of AD includes the inability to recall vocabulary, loss of reading and writing skills, impairment of coordination of complex motor sequences resulting for example in an increased risk of falling, urinary incontinence, impairment of long-term memory, illusionary misidentifications and other delusional symptoms. Advanced symptoms of AD include reduction of language to simple phrases or even single words, eventually leading to complete loss of speech, severe reduction in muscle mass and mobility and loss of bodily functions.

AD is considered to be the cause of 60% to 70% of cases of dementia. Behavioral and psychological symptoms of dementia are considered to constitute a major clinical component of AD (Robert 2005, European Psychiatry 20.7: 490-496). Although the speed of progression of AD can vary, the average life expectancy following diagnosis of AD is about three to nine years (Todd 2013, International journal of geriatric psychiatry 28.11: 1109-1124).

The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Dementia as referred to herein includes a variety of brain diseases that cause a decrease in the ability to think and remember, often accompanied with language and motor skill problems. As mentioned above, the most common type of dementia is Alzheimer's disease. Other types include, for example, vascular dementia, Lewy body dementia, frontotemporal dementia, normal pressure hydrocephalus, Parkinson's disease, syphilis, and Creutzfeldt-Jakob disease. Known risk factors for developing dementia include high blood pressure, smoking, diabetes, and obesity. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Leukodystrophies are a group of disorders that are characterized by degeneration of the white matter in the brain. Leukodystrophies are thought to be caused by imperfect growth or development of the myelin sheath or by loss of myelin due to inflammation in the central nervous system. The degeneration of white matter can be seen in an MRI and used to diagnose leukodystrophies (Cheon 2002, Radiographics 22.3: 461-476). Symptoms of leukodystrophies are usually dependent on the age of onset, which is predominantly in infancy and early childhood, and including decreased motor function, muscle rigidity, impairment of sight and hearing, ataxia and mental retardation. Leukodystrophy disorders include, for example, X-linked adrenoleukodystrophy, Krabbe Disease, Metachromatic Leukodystrophy (MLD), Canavan Disease and Alexander Disease. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Autism spectrum disorder (ASD) is characterized by a group of complex neurological and developmental disorders. ASD affects the structure and function of the brain and nervous system. Typical characteristics of ASD include social problems such as difficulty in communicating and interacting with others, repetitive behaviors, limited interests or activities and facial expressions, movements, gestures that do not match what is being said. According to the Centers for Disease Control and Prevention (CDC) around 1 in 68 children has been identified with some form of ASD. The diagnosis of ADS may be difficult and is commonly based on the Diagnostic and Statistical Manual of Mental Disorders (DSM). In the past, Asperger's syndrome and Autistic Disorder were considered to be separate disorders. However, in May 2013, a new version of the Diagnostic and Statistical Manual of Mental Disorders (DSM-5), the common manual from the American Psychiatric Association used to diagnose different mental health conditions, was released. The DSM-5 manual now only includes the range of characteristics and severity within one category, called Autism Spectrum Disorder (ASD), and does not highlight subcategories of a larger disorder anymore (previous subcategories were: Autistic disorder, Asperger syndrome, Childhood disintegrative disorder, Pervasive developmental disorder not otherwise specified). According to DSM-5 guidelines, people whose symptoms were previously diagnosed as Asperger's syndrome or Autistic Disorder are now included as part of the category called Autism Spectrum Disorder (ASD). The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Attention-deficit disorders, also referred to as attention deficit disorder (ADD) or Attention deficit hyperactivity disorder (ADHD), refer to a group of neurodevelopmental disorders.

According to the newest new version of the Diagnostic and Statistical Manual of Mental Disorders (DSM-5), several symptoms must be present before age 12 for the diagnosis of Attention-deficit disorders. Typical symptoms of ADD or ADHD include symptoms of inattention such as difficulties following instructions or organizing tasks, symptoms of hyperactivity or impulsivity such as difficulties in remaining seated or awaiting turns (e.g., answering before questions have been completed, interruption of conversations). The method of the present disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Intellectual disabilities as defined by DSM-5. Intellectual disability (intellectual developmental disorder) as a DSM-5 diagnostic term replaces “mental retardation” used in previous editions of the manuals. In DSM-5, the diagnosis of intellectual disability (intellectual developmental disorder) is revised from the DSM-IV diagnosis of mental retardation (American Psychiatric Association. Diagnostic and statistical manual of mental disorders (DSM-5®). American Psychiatric Pub, 2013.) The revised disorder reflects the manual's move away from a multiaxial approach to evaluating conditions. Intellectual disability as defined by DSM-5 involves impairments of general mental abilities that impact adaptive functioning in three domains, or areas: (1) the conceptual domain includes skills in language, reading, writing, math, reasoning, knowledge, and memory; (2) the social domain refers to empathy, social judgment, interpersonal communication skills, the ability to make and retain friendships, and similar capacities; (3) the practical domain centers on self-management in areas such as personal care, job responsibilities, money management, recreation, and organizing school and work tasks. While intellectual disability does not have a specific age requirement, an individual's symptoms must begin during the developmental period and are diagnosed based on the severity of deficits in adaptive functioning. The disorder is considered chronic and often co-occurs with other mental conditions like depression, attention-deficit/hyperactivity disorder, and autism spectrum disorder. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Impairment of cognitive performances and reserve related to aging refers to any age-related decline of cognitive performance such as the ability to think and remember and/or any age-related effects on brain size (also referred as “brain reserve”) or neural count (also referred to as “cognitive reserve”). Cognitive decline, for example in speeded abilities, executive function, and memory, is believed to typify normal aging (Gunstad 2006, Journal of Geriatric Psychiatry and Neurology 19.2: 59-64). The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Parkinson's disease (PD) is a progressive disorder of the central nervous system that mainly affects the motor system. Typical symptoms include shaking, rigidity, slowness of movement, difficulty with walking. Other symptoms including sensory, sleep, and emotional problems as well as thinking and behavioral problems may also occur as well as depression and anxiety issues are commonly observed in the advanced stages of the disease. The cause of Parkinson's disease is currently unknown, but the motor symptoms of the disease are thought to result from the death of cells in the substantia nigra leading to a decrease in dopamine in these areas. However, some of the non-motor symptoms are often present at the time of diagnosis and can precede motor symptoms. Diagnosis of PD is mainly based on the clinical assessment of symptoms combined with other tests such as neuroimaging being used to rule out other diseases. The occurrence of Parkinson's disease is most common in people over the age of 60, affecting males more often than females. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Huntington's disease (HD), also referred to as Huntington's chorea, is an inherited disorder caused by an autosomal dominant mutation in the Huntingtin gene (HTT). HD is a fatal disease caused by death of brains cells. Symptoms of Huntington's disease can begin at any age from infancy to old age, although the usually become noticeable between the age of 35 and 44 years. Early symptoms include changes in personality, cognition, and physical skills (Walker 2007, The Lancet 369.9557: 218-228). The most characteristic initial physical symptoms are random and uncontrollable movements referred to as chorea. Further symptoms include seizures, abnormal facial expression, difficulties in chewing, swallowing and speaking. Diagnosis of HD is usually based on the clinical assessment of symptoms as well as genetic testing. The method of the present disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Amyotrophic lateral sclerosis (ALS), also known as the most frequent form of motor neuron disease (MND), is a late-onset fatal neurodegenerative disease affecting motor neurons. ALS occurs with an incidence of about 1/100,000. Most ALS cases are sporadic, but 5-10% of the cases are familial ALS. Both sporadic and familial ALS (FALS) are associated with degeneration of cortical and spinal motor neurons. Typical symptoms include muscle weakness and atrophy throughout the body, impairment of cognitive functions. The diagnosis of ALS commonly includes a clinical examination and series of diagnostic tests, often ruling out other diseases that mimic ALS. For ALS to be diagnosed, usually symptoms of both upper and lower motor neuron damage that cannot be attributed to other causes must be present. The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

Neuroleptic malignant syndrome (NMS) is a life-threatening neurological disorder most often caused by an adverse reaction to neuroleptic or antipsychotic drugs such as haloperidol, droperidol, promethazine, chlorpromazine, clozapine, olanzapine, risperidone, quetiapine, or ziprasidone. Symptoms include muscle cramps, tremors, fever, symptoms of autonomic nervous system instability such as unstable blood pressure, and alterations in mental status (agitation, delirium, or coma). The muscular symptoms in NMS are most likely caused by blockade of the dopamine receptor D2, leading to abnormal function of the basal ganglia similar to that seen in Parkinson's disease. Moreover, an elevated level of plasma creatine kinase is associated with NMS (Strawn 2007, American Journal of Psychiatry 164.6: 870-876). The method of this disclosure can be typically also applied mutatis mutandis for those purposes referred to in accordance with MS. In particular, the method may be applied for assessing the disease including the aspects described elsewhere in detail, making risk assessments, more typically, establishing risk prediction models and/or developing algorithmic solutions using for instance machine-learning and pattern recognition techniques.

The term “subject” as used herein refers to animals and, typically, to mammals. In particular, the subject is a primate and, most typically, a human. The subject in accordance with this disclosure shall suffer from or shall be suspected to suffer from a cognition and movement disease or disorder, i.e., it may already show some or all of the symptoms associated with the said disease.

The term “cognition and/or fine motoric activity parameter” as used herein refers to a parameter which is indicative for the capability of a subject to perform a certain cognitive task or fine motoric physical activity, in particular, movement and/or cognitive capabilities required for carrying out or for the coordination of movement activities. Typically, said movement is movement of the hands or parts thereof such as individual fingers, i.e., hand motoric functions. Depending on the type of activity which is measured, the cognition and/or fine motoric activity parameter can be derived from the dataset acquired by the activity measurement performed on the subject. Such performance parameters may be based on the time which is required to perform a certain activity, e.g., it may be the velocity or frequency with which a certain activity is performed or it may be the duration of the gap between activities. Further, it may be based on the accuracy with which a task is performed or may be based on the amount of task that can be performed. Particular cognition and/or fine motoric activity parameters to be used in accordance with this disclosure depend on the measured activity and are listed elsewhere herein in more detail.

The term “at least one” means that one or more parameters, such as fine motoric activity parameters, may be determined in accordance with this disclosure, i.e., at least two, at least three, at least four, at least five, at least six, at least seven, at least eight at least nine or at least ten or even more different parameters. Thus, there is no upper limit for the number of different parameters which can be determined in accordance with the method of this disclosure. Typically, however, there will be between one and three different parameters per dataset of activity measurement determined.

The term “dataset of activity measurements” refers, in principle, to the entirety of data acquired by the mobile device from a subject during activity measurements or any subset of said data useful for deriving a cognition and/or fine motoric activity parameter. Details are also found elsewhere herein. In particular, the activity measurements in connection with the term “dataset of cognition and/or fine motoric activity measurements” as used in accordance with the present disclosure comprise measurements of datasets during performances of a Symbol Digit Modalities Test (eSDMT), a Draw a Shape test and/or a Squeeze a Shape test as described elsewhere herein in detail. Typically, the cognition and/or fine motoric activity measured by these respective tests is attention, information processing speed, visual scanning, and/or hand motoric activity.

In the following, particular envisaged activity tests and means for measuring by a mobile device in accordance with the method of the present disclosure are specified:

(1) A computer-implemented (electronic) Symbol Digit Modalities Test (eSDMT).

In an embodiment, the mobile device is, thus, adapted for performing or acquiring a data from an electronic Symbol Digit Modalities Test (eSDMT). The conventional paper SDMT version of the test consists of a sequence of 120 symbols to be displayed in a maximum 90 seconds and a reference key legend (3 versions are available) with 9 symbols in a given order and their respective matching digits from 1 to 9. The smartphone-based eSDMT is meant to be self-administered by patients and will use as sequence of symbols, typically, the same sequence of 110 symbols, and a random alternation (form one test to the next) between reference key legends, typically the 3 reference key legends of the paper/oral version of SDMT. The eSDMT, similar to the paper/oral version, measures the speed (number of correct paired responses) to pair abstract symbols with specific digits in a predetermined time window, such as 90 seconds time. The test is, typically, performed weekly but could alternatively be performed at higher (e.g., daily) or lower (e.g., bi-weekly) frequency. The test could also alternatively encompass more than 110 symbols and more and/or evolutionary versions of reference key legends. The symbol sequence could also be administered randomly or according to any other modified pre-specified sequence.

Typical eSDMT performance parameters of interest:

1. Number of correct responses
a. Total number of overall correct responses (CR) in 90 seconds (similar to oral/paper SDMT)
b. Number of correct responses from time 0 to 30 seconds (CR₀₋₃₀)
c. Number of correct responses from time 30 to 60 seconds (CR₃₀₋₆₀)
d. Number of correct responses from time 60 to 90 seconds (CR₆₀₋₉₀)
e. Number of correct responses from time 0 to 45 seconds (CR₀₋₄₅)
f. Number of correct responses from time 45 to 90 seconds (CR₄₅₋₉₀)
g. Number of correct responses from time i to j seconds (CRᵢ₋ⱼ), where i, j are between 1 and 90 seconds and i<j

2. Number of errors
a. Total number of errors (E) in 90 seconds
b. Number of errors from time 0 to 30 seconds (E₀₋₃₀)
c. Number of errors from time 30 to 60 seconds (E₃₀₋₆₀)
d. Number of errors from time 60 to 90 seconds (E₆₀₋₉₀)
e. Number of errors from time 0 to 45 seconds (E₀₋₄₅)
f. Number of errors from time 45 to 90 seconds (E₄₅₋₉₀)
g. Number of errors from time i to j seconds (Eᵢ₋ⱼ), where i, j are between 1 and 90 seconds and i<j

3. Number of responses
a. Total number of overall responses (R) in 90 seconds
b. Number of responses from time 0 to 30 seconds (R₀₋₃₀)
c. Number of responses from time 30 to 60 seconds (R₃₀₋₆₀)
d. Number of responses from time 60 to 90 seconds (R₆₀₋₉₀)
e. Number of responses from time 0 to 45 seconds (R₀₋₄₅)
f. Number of responses from time 45 to 90 seconds (R₄₅₋₉₀)

4. Accuracy rate
a. Mean accuracy rate (AR) over 90 seconds: AR=CR/R
b. Mean accuracy rate (AR) from time 0 to 30 seconds: AR₀₋₃₀=CR₀₋₃₀/R₀₋₃₀
c. Mean accuracy rate (AR) from time 30 to 60 seconds: AR₃₀₋₆₀=CR₃₀₋₆₀/R₃₀₋₆₀
d. Mean accuracy rate (AR) from time 60 to 90 seconds: AR₆₀₋₉₀=CR₆₀₋₉₀/R60-90
e. Mean accuracy rate (AR) from time 0 to 45 seconds: AR₀₋₄₅=CR₀₋₄₅/R₀₋₄₅
f. Mean accuracy rate (AR) from time 45 to 90 seconds: AR₄₅₋₉₀=CR₄₅₋₉₀/R₄₅₋₉₀

5. End of task fatigability indices
a. Speed Fatigability Index (SFI) in last 30 seconds: SFI₆₀₋₉₀=CR₆₀₋₉₀/max (CR₀₋₃₀, CR₃₀₋₆₀)
b. SFI in last 45 seconds: SFI₄₅₋₉₀=CR₄₅₋₉₀/CR₀₋₄₅
c. Accuracy Fatigability Index (AFI) in last 30 seconds: AFI₆₀₋₉₀=AR₆₀₋₉₀/max (AR₀₋₃₀, AR₃₀₋₆₀)
d. AFI in last 45 seconds: AFI₄₅₋₉₀=AR₄₅₋₉₀/AR₀₋₄₅

6. Longest sequence of consecutive correct responses
a. Number of correct responses within the longest sequence of overall consecutive correct responses (CCR) in 90 seconds
b. Number of correct responses within the longest sequence of consecutive correct responses from time 0 to 30 seconds (CCR₀₋₃₀)
c. Number of correct responses within the longest sequence of consecutive correct responses from time 30 to 60 seconds (CCR₃₀₋₆₀)
d. Number of correct responses within the longest sequence of consecutive correct responses from time 60 to 90 seconds (CCR₆₀₋₉₀)
e. Number of correct responses within the longest sequence of consecutive correct responses from time 0 to 45 seconds (CCR₀₋₄₅)
f. Number of correct responses within the longest sequence of consecutive correct responses from time 45 to 90 seconds (CCR₄₅₋₉₀)

7. Time Gap between responses
a. Continuous variable analysis of gap (G) time between two successive responses
b. Maximal gap (GM) time elapsed between two successive responses over 90 seconds
c. Maximal gap time elapsed between two successive responses from time 0 to 30 seconds (GM₀₋₃₀)
d. Maximal gap time elapsed between two successive responses from time 30 to 60 seconds (GM₃₀₋₆₀)
e. Maximal gap time elapsed between two successive responses from time 60 to 90 seconds (GM₆₀₋₉₀)
f. Maximal gap time elapsed between two successive responses from time 0 to 45 seconds (GM₀₋₄₅)
g. Maximal gap time elapsed between two successive responses from time 45 to 90 seconds (GM₄₅₋₉₀)

8. Time Gap between correct responses
a. Continuous variable analysis of gap (Gc) time between two successive correct responses
b. Maximal gap time elapsed between two successive correct responses (GcM) over 90 seconds
c. Maximal gap time elapsed between two successive correct responses from time 0 to 30 seconds (GcM₀₋₃₀)
d. Maximal gap time elapsed between two successive correct responses from time 30 to 60 seconds (GcM₃₀₋₆₀)
e. Maximal gap time elapsed between two successive correct responses from time 60 to 90 seconds (GcM₆₀₋₉₀)
f. Maximal gap time elapsed between two successive correct responses from time 0 to 45 seconds (GcM₀₋₄₅)
g. Maximal gap time elapsed between two successive correct responses from time 45 to 90 seconds (GcM₄₅₋₉₀)

9. Fine finger motor skill function parameters captured during eSDMT
a. Continuous variable analysis of duration of touchscreen contacts (Tts), deviation between touchscreen contacts (Dts) and center of closest target digit key, and mistyped touchscreen contacts (Mts) (i.e. contacts not triggering key hit or triggering key hit but associated with secondary sliding on screen), while typing responses over 90 seconds
b. Respective variables by epochs from time 0 to 30 seconds: Tts₀₋₃₀, Dts₀₋₃₀, Mts₀₋₃₀
c. Respective variables by epochs from time 30 to 60 seconds: Tts₃₀₋₆₀, Dts₃₀₋₆₀, Mts₃₀₋₆₀
d. Respective variables by epochs from time 60 to 90 seconds: Tts₆₀₋₉₀, Dts₆₀₋₉₀, Mts₆₀₋₉₀
e. Respective variables by epochs from time 0 to 45 seconds: Tts₀₋₄₅, Dts₀₋₄₅, Mts₀₋₄₅
f. Respective variables by epochs from time 45 to 90 seconds: Tts₄₅₋₉₀, Dts₄₅₋₉₀, Mts₄₅₋₉₀

10. Symbol-specific analysis of performances by single symbol or cluster of symbols
a. CR for each of the 9 symbols individually and all their possible clustered combinations
b. AR for each of the 9 symbols individually and all their possible clustered combinations
c. Gap time (G) from prior response to recorded responses for each of the 9 symbols individually and all their possible clustered combinations
d. Pattern analysis to recognize preferential incorrect responses by exploring the type of mistaken substitutions for the 9 symbols individually and the 9 digit responses individually

11. Learning and cognitive reserve analysis
a. Change from baseline (baseline defined as the mean performance from the first 2 administrations of the test) in CR (overall and symbol-specific as described in 9) between successive administrations of eSDMT
b. Change from baseline (baseline defined as the mean performance from the first 2 administrations of the test) in AR (overall and symbol-specific as described in 9) between successive administrations of eSDMT
c. Change from baseline (baseline defined as the mean performance from the first 2 administrations of the test) in mean G and GM (overall and symbol-specific as described in 9) between successive administrations of eSDMT
d. Change from baseline (baseline defined as the mean performance from the first 2 administrations of the test) in mean Gc and GcM (overall and symbol-specific as described in 9) between successive administrations of eSDMT
e. Change from baseline (baseline defined as the mean performance from the first 2 administrations of the test) in SFI₆₀₋₉₀ and SFI₄₅₋₉₀ between successive administrations of eSDMT
f. Change from baseline (baseline defined as the mean performance from the first 2 administrations of the test) in AFI₆₀₋₉₀ and AFI₄₅₋₉₀ between successive administrations of eSDMT
g. Change from baseline (baseline defined as the mean performance from the first 2 administrations of the test) in Tts between successive administrations of eSDMT
h. Change from baseline (baseline defined as the mean performance from the first 2 administrations of the test) in Dts between successive administrations of eSDMT
i. Change from baseline (baseline defined as the mean performance from the first 2 administrations of the test) in Mts between successive administrations of eSDMT
   (2) A computer-implemented test evaluating fine motoric capabilities (fine motoric assessments), in particular, hand motor functions and, in particular, the touchscreen-based “Draw a Shape” and “Squeeze a Shape” tests.

In yet another embodiment, the mobile device is adapted to perform or acquire data from fine motoric assessments and, in particular, Hand Motor Function Tests. Manual dexterity (hand motor function) characterizes an individual's ability to coordinate movement of the hand and fingers and manipulate objects in a timely manner. Manual dexterity greatly impacts a subject's performance in daily activities, completing work related tasks, and engaging in leisure activities.

Manual dexterity was identified in 2007 as a core construct for inclusion in the National Institutes of Health Toolbox (NIH) Toolbox for the assessment of neurological and behavioral function, as part of the NIH Blueprint for Neuroscience Research initiative, which developed brief yet comprehensive instruments to measure motor, cognitive, sensory, and emotional function. After reviewing existing measures, experts recommended two candidate measures of manual dexterity: 1) 9-Hole Peg Test (9HPT), and 2) Grooved Pegboard Test (GPT) for potential inclusion in the NIH Toolbox because of their applicability across the life span, psychometric soundness, brevity (completion time for one trial is relatively short), and applicability in diverse settings.

Primarily, the 9HPT was selected because it met the most inclusion criteria and the test was easy to administer in all age groups, especially younger children. The time to administer the 9-hole peg test was brief (<5 min to measure for both hands) as required for inclusion in the NIH Toolbox. Existing literature supported 9HPT as a reliable and valid measure of finger dexterity, and as capable for assessing hand dexterity in various diagnostic groups (i.e., multiple sclerosis, stroke, cerebral palsy, cerebellar impairment, and Parkinson's disease).

Normative data for the 9HPT have been published across the age span including children and elderly adults and, since the late 90s, 9HPT represents the key component of functional upper limb assessment from the Multiple Sclerosis Functional Composite (MSFC) scale.

Moreover, in accordance with this disclosure, two touchscreen-based application tests “Draw a Shape” and “Squeeze a Shape” were developed that aimed at replicating on a user-friendly mobile device interface the characteristics of 9HPT and GPT for enabling remote self-assessment of hand motor function in neurological disorders. The “Draw a Shape” and “Squeeze a Shape” tests will evaluate upper limb motor function and manual dexterity (pinching, drawing) and will be sensitive to change and abnormalities in pyramidal, extrapyramidal, sensory and cerebellar components of upper limb nervous system but also to neuromuscular and myogenic alteration of upper limb function. . . . The test is, typically, performed daily but could alternatively be performed at lower (e.g., weekly or bi-weekly) frequency.

The aim of the “Draw a Shape” test is to assess fine finger control and stroke sequencing. The test is considered to cover the following aspects of impaired hand motor function: tremor and spasticity and impaired hand-eye coordination. The patients are instructed to hold the mobile device in the untested hand and draw on a touchscreen of the mobile device 6 pre-written alternating shapes of increasing complexity (linear, rectangular, circular, sinusoidal, and spiral; vide infra) with the second finger of the tested hand “as fast and as accurately as possible” within a maximum time of for instance 30 seconds. To draw a shape successfully the patient's finger has to slide continuously on the touchscreen and connect indicated start and end points passing through all indicated check points and keeping within the boundaries of the writing path as much as possible. The patient has maximum two attempts to successfully complete each of the 6 shapes. The test will be alternatingly performed with right and left hand. The user will be instructed on daily alternation. The two linear shapes have each a specific number “a” of checkpoints to connect, i.e., “a-1” segments. The square shape has a specific number “b” of checkpoints to connect, i.e., “b-1” segments. The circular shape has a specific number “c” of checkpoints to connect, i.e., “c-1” segments. The eight-shape has a specific number “d” of checkpoints to connect, i.e., “d-1” segments. The spiral shape has a specific number “e” of checkpoints to connect, “e-1” segments. Completing the 6 shapes then implies to draw successfully a total of “(2a+b+c+d+e-6)” segments.

Typical Draw a Shape test performance parameters of interest:
Based on shape complexity, the linear and square shapes can be associated with a weighting factor (Wf) of 1, circular and sinusoidal shapes a weighting factor of 2, and the spiral shape a weighting factor of 3. A shape which is successfully completed on the second attempt can be associated with a weighting factor of 0.5. These weighting factors are numerical examples which can be changed in the context of the present disclosure.

1. Shape completion performance scores:
a. Number of successfully completed shapes (0 to 6) (ΣSh) per test
b. Number of shapes successfully completed at first attempt (0 to 6) (ΣSh₁)
c. Number of shapes successfully completed at second attempt (0 to 6) (ΣSh₂)
d. Number of failed/uncompleted shapes on all attempts (0 to 12) (ΣF)
e. Shape completion score reflecting the number of successfully completed shapes adjusted with weighting factors for different complexity levels for respective shapes (0 to 10) (Σ[Sh*Wf])
f. Shape completion score reflecting the number of successfully completed shapes adjusted with weighting factors for different complexity levels for respective shapes and accounting for success at first vs. second attempts (0 to 10) (Σ[Sh₁*Wf]+Σ[Sh₂*Wf*0.5])
g. Shape completion scores as defined in 1(e), and 1(f) may account for speed at test completion if being multiplied by 30/t, where t would represent the time in seconds to complete the test.
h. Overall and first attempt completion rate for each 6 individual shapes based on multiple testing within a certain period of time: (ΣSh₁)/ (ΣSh₁+ΣSh₂+ΣF) and (ΣSh₁+ΣSh₂)/(ΣSh₁+ΣSh₂+ΣF)

2. Segment completion and celerity performance scores/measures:
(analysis based on best of two attempts [highest number of completed segments] for each shape, if applicable)
a. Number of successfully completed segments (0 to [2a+b+c+d+e-6]) (ΣSe) per test
b. Mean celerity ([C], segments/second) of successfully completed segments: C=ΣSe/t, where t would represent the time in seconds to complete the test (max 30 seconds)
c. Segment completion score reflecting the number of successfully completed segments adjusted with weighting factors for different complexity levels for respective shapes (Σ[Se*Wf])
d. Speed-adjusted and weighted segment completion score (Σ[Se*Wf]*30/t), where t would represent the time in seconds to complete the test.
e. Shape-specific number of successfully completed segments for linear and square shapes (ΣSe_{LS})
f. Shape-specific number of successfully completed segments for circular and sinusoidal shapes (ΣSe_{CS})
g. Shape-specific number of successfully completed segments for spiral shape (ΣSe_{S})
h. Shape-specific mean linear celerity for successfully completed segments performed in linear and square shape testing: C_{L}=ΣSe_{LS}/t, where t would represent the cumulative epoch time in seconds elapsed from starting to finishing points of the corresponding successfully completed segments within these specific shapes
i. Shape-specific mean circular celerity for successfully completed segments performed in circular and sinusoidal shape testing: C_{C}=ΣSe_{CS}/t, where t would represent the cumulative epoch time in seconds elapsed from starting to finishing points of the corresponding successfully completed segments within these specific shapes
j. Shape-specific mean spiral celerity for successfully completed segments performed in the spiral shape testing: C_{S}=ΣSe_{S}/t, where t would represent the cumulative epoch time in seconds elapsed from starting to finishing points of the corresponding successfully completed segments within this specific shape

3. Drawing precision performance scores/measures:
(analysis based on best of two attempts[highest number of completed segments] for each shape, if applicable)
a. Deviation (Dev) calculated as the sum of overall area under the curve (AUC) measures of integrated surface deviations between the drawn trajectory and the target drawing path from starting to ending checkpoints that were reached for each specific shapes divided by the total cumulative length of the corresponding target path within these shapes (from starting to ending checkpoints that were reached)
b. Linear deviation (Dev_{L}) calculated as Dev in 3(a) but specifically from the linear and square shape testing results.
c. Circular deviation (Dev_{C}) calculated as Dev in 3(a) but specifically from the circular and sinusoidal shape testing results.
d. Spiral deviation (Dev_{S}) calculated as Dev in 3(a) but specifically from the spiral shape testing results.
e. Shape-specific deviation (Dev₁₋₆) calculated as Dev in 3(a) but from each of the 6 distinct shape testing results separately, only applicable for those shapes where at least 3 segments were successfully completed within the best attempt.
f. Continuous variable analysis of any other methods of calculating shape-specific or shape-agnostic overall deviation from the target trajectory.

The aim of the Squeeze a Shape test is to assess fine distal motor manipulation (gripping and grasping) and control by evaluating accuracy of pinch closed finger movement. The test is considered to cover the following aspects of impaired hand motor function: impaired gripping/grasping function, muscle weakness, and impaired hand-eye coordination. The patients are instructed to hold the mobile device in the untested hand and by touching the screen with two fingers from the same hand (thumb+second or thumb+third finger preferred) to squeeze/pinch as many round shapes (i.e., tomatoes) as they can during 30 seconds. Impaired fine motor manipulation will affect the performance. Test will be alternatingly performed with right and left hand. User will be instructed on daily alternation.

Typical Squeeze a Shape test performance parameters of interest:

1. Number of squeezed shapes
a. Total number of tomato shapes squeezed in 30 seconds (ΣSh)
b. Total number of tomatoes squeezed at first attempt (ΣSh₁) in 30 seconds (a first attempt is detected as the first double contact on screen following a successful squeezing if not the very first attempt of the test) 2. Pinching precision measures:
a. Pinching success rate (P_{SR}) defined as ΣSh divided by the total number of pinching (ΣP) attempts (measured as the total number of separately detected double finger contacts on screen) within the total duration of the test.
b. Double touching asynchrony (DTA) measured as the lag time between first and second fingers touch the screen for all double contacts detected.
c. Pinching target precision (P_{TP}) measured as the distance from midpoint between the starting touch points of the two fingers at double contact to the center of the tomato shape, for all double contacts detected.
d. Pinching finger movement asymmetry (P_{FMA}) measured as the ratio between respective distances slid by the two fingers (shortest/longest) from the double contact starting points until reaching pinch gap, for all double contacts successfully pinching.
e. Pinching finger velocity (P_{FV}) measured as the speed (mm/sec) of each one and/or both fingers sliding on the screen from time of double contact until reaching pinch gap, for all double contacts successfully pinching.
f. Pinching finger asynchrony (P_{FA}) measured as the ratio between velocities of respective individual fingers sliding on the screen (slowest/fastest) from the time of double contact until reaching pinch gap, for all double contacts successfully pinching.
g. Continuous variable analysis of 2a to 2f over time as well as their analysis by epochs of variable duration (5-15 seconds)
h. Continuous variable analysis of integrated measures of deviation from target drawn trajectory for all tested shapes (in particular the spiral and square)

It will be understood that the mobile device to be applied in accordance with this disclosure may be adapted to perform one or more of the aforementioned activity tests. In particular, it may be adapted to perform one, two or all three of these tests. Typically, combinations of tests may be implemented on the mobile device.

Moreover, in the method of this disclosure at least one further parameter may be determined from an activity dataset obtained from the mobile device. The said further parameter being typically a performance parameter which is indicative for the capability of a subject to perform a certain physical or cognitive activity, in particular, it is a parameter indicative for the subject's motoric and/or fine motoric capabilities, color vision, attention, dexterity and/or cognitive capabilities. Depending on the type of activity which is measured, the performance parameter can be derived from the dataset acquired by the activity measurement performed on the subject. Such performance parameters may be based on the time which is required to perform a certain activity, e.g., it may be the velocity or frequency with which a certain activity is performed or it may be the duration of the gap between activities. Further, it may be based on the accuracy with which a task is performed or may be based on the amount of task that can be performed.

Particular performance parameters to be used in accordance with this disclosure depend on the measured activity and are listed elsewhere herein in more detail. The dataset of activity measurements referred to in this context relates to the entirety of data acquired by the mobile device from a subject during activity measurements or any subset of said data useful for deriving the performance parameter. This depends also on the cognition and movement disease or disorder to be assessed. In the case of MS, activities to be performed and measured by the mobile device during performance are, typically, performing active walking tests, in particular, the 2-Minute Walking Test (2MWT) and the Five U-Turn Test (5UTT), passive continuous analysis of gait (CAG), performing orthostatic posture and balance tests, in particular, the Static Balance Test (SBT), answering mood scale questions, answering questions on quality of life and disease symptoms, in particular, by performing the 29-Item Multiple Sclerosis Impact Scale (MSIS29) questionnaire and/or the Multiple Sclerosis Symptom Tracker (MSST). Moreover, the dataset of activity measurements may be obtained from a passive monitoring of all or a predetermined subset of activities of a subject performed during a certain time window, e.g., during daily routine. These measurements allow for assessing a subject's quality of life, fatigue, mental state and/or mood. In this context, passive monitoring may include continuous measurements of gait, the amount of movement in daily routines in general, e.g., frequency and/or velocity of walking, the types of movement in daily routines, e.g., amount, ability and/or velocity to stand up/sit down, stand still and balance, general mobility in daily living as indicated by, e.g., visiting more or fewer locations, changes in moving behavior as indicated by, e.g., changes in types of locations visited.

Thus, the mobile device may be adapted to perform further cognition and movement disorder and disease tests such as computer-implemented versions of active walking tests, in particular, the 2-Minute Walking Test (2MWT) and the Five U-Turn Test (5UTT), passive continuous analysis of gait (CAG), orthostatic posture and balance tests, in particular, the Static Balance Test (SBT), mood scale questions, questions on quality of life, in particular, by the 29-Item Multiple Sclerosis Impact Scale (MSIS29) questionnaire, the Multiple Sclerosis Symptom Tracker (MSST) and/or passive monitoring of all or a predetermined subset of activities of a subject performed during a certain time window.

In the following, particular envisaged activity tests and means for measuring by a mobile device in accordance with the method of the present disclosure are specified:

(3) A sensor-based (e.g., accelerometer, gyroscope, magnetometer, global positioning system [GPS]) and computer implemented test for measures of ambulation performance and gait and stride dynamics, in particular, the 2-Minute Walking Test (2MWT) and the Five U-Turn Test (5UTT), and test for ambulation performance, step/stride dynamics, and upper limb motor function while walking using data collected from passive Continuous Analysis of Gait (CAG).

In one embodiment, the mobile device is adapted to perform or acquire data from the Two-Minute Walking Test (2MWT). The aim of this test is to assess difficulties, fatigability or unusual patterns in long-distance walking by capturing gait features in a two-minute walk test (2MWT). Data will be captured from the mobile device. A decrease of stride and step length, increase in stride duration, increase in step duration and asymmetry and less periodic strides and steps may be observed in case of disability progression or emerging relapse. Arm swing dynamic while walking will also be assessed via the mobile device. The subject will be instructed to “walk as fast and as long as you can for 2 minutes but walk safely.” The 2MWT is a simple test that is required to be performed indoor or outdoor, on even ground in a place where patients have identified they could walk straight for as far as >200 meters without U-turns. Subjects are allowed to wear regular footwear and an assistive device and/or orthotic as needed. The test is typically performed daily.

Typical 2MWT performance parameters of particular interest:

1. Surrogate of walking speed and spasticity:
a. Total number of steps detected in, e.g., 2 minutes (ΣS)
b. Total number of rest stops if any detected in 2 minutes (ΣRs)
c. Continuous variable analysis of walking step time (WsT) duration throughout the 2MWT
d. Continuous variable analysis ofwalking step velocity (WsV) throughout the 2MWT (step/second)
e. Step asymmetry rate throughout the 2MWT (mean difference of step duration between one step to the next divided by mean step duration): SAR=meanΔ(WsTₓ−WsTₓ₊₁)/(120/ΣS)
f. Total number of steps detected for each epoch of 20 seconds (ΣS_{t, t+20})
g. Mean walking step time duration in each epoch of 20 seconds: WsT_{t, t+20}=20/ΣS_{t, t+20}
h. Mean walking step velocity in each epoch of 20 seconds: WsV_{t, t+20}=ΣS_{t, t+20}/20
i. Step asymmetry rate in each epoch of 20 seconds: SAR_{t, t+20}=meanΔ_{t, t+20}(WsTₓ−WsTₓ₊₁)/(20/ΣS_{t, t+20})
j. Step length and total distance walked through biomechanical modelling 2. Walking fatigability indices:
a. Deceleration index: DI=WsV₁₀₀₋₂₀₀/max (WsV₀₋₂₀, WsV₂₀₋₄₀, WsV₄₀₋₆₀)
b. Asymmetry index: AI=SAR₁₀₀₋₁₂₀/min (SAR₀₋₂₀, SAR₂₀₋₄₀, SAR₄₀₋₆₀)

In another embodiment, the mobile device is adapted to perform or acquire data from the Five U-Turn Test (5UTT). The aim of this test is to assess difficulties or unusual patterns in performing U-turns while walking on a short distance at comfortable pace. The 5UTT is required to be performed indoor or outdoor, on even ground where patients are instructed to “walk safely and perform five successive U-turns going back and forward between two points a few meters apart.” Gait feature data (change in step counts, step duration and asymmetry during U-turns, U-turn duration, turning speed and change in arm swing during U-turns) during this task will be captured by the mobile device. Subjects are allowed to wear regular footwear and an assistive device and/or orthotic as needed. The test is typically performed daily.

Typical 5UTT performance parameters of interest:

1. Mean number of steps needed from start to end of complete U-turn (ΣSu)

2. Mean time needed from start to end of complete U-turn (Tu)

3. Mean walking step duration: Tsu=Tu/ΣSu

4. Turn direction (left/right)

5. Turning speed (degrees/sec)

In yet another embodiment, the mobile device is adapted for performing or acquiring data from Continuous Analysis of Gait (CAG). Continuous recording of gait feature data (step counts, duration, and asymmetry, as well as arm swing dynamic while walking) captured from sensors will allow passive monitoring of daily volume and quality of walking dynamics. Activity detection is a prior step to gait detection and analysis and activity analysis. It may be based on different more or less complex approaches (Rai, 2012, Zee: zero-effort crowdsourcing for indoor localization, Proceedings of the 18th annual international conference on Mobile computing and networking, ACM; Alsheikh, M. A., Selim, A., Niyato, D., Doyle, L., Lin, S., & Tan, H.-P, 2015, Deep Activity Recognition Models with Triaxial Accelerometers, arXiv preprint arXiv:1511.04664; or Ordóñez, F. J. & Roggen, D., 2016, Deep Convolutional and LSTM Recurrent Neural Networks for Multimodal Wearable Activity Recognition, Sensors, 16(1), 115), which considers windows of one second as active if the standard deviation of the accelerometer signal is above 0.01 g. The test is typically performed daily.

Typical CAG performance parameters of interest:
Surrogate of daily walking range and speed:
a. Total number of steps detected for each day of active recording (ΣSd)
b. Total cumulative time of detected walking for each day of active recording (ΣT)
c. Total number of intervals of continuous walking for each day of active recording (ΣId)
d. Frequency distribution of the number of steps detected within each interval of continuous walking for each day of active recording (ΔSi)
e. Maximal number of steps in a single interval of continuous walking for each day of active recording (Scmax)
f. Mean walking step time duration for each day of active recording: WsT=ΣT/ΣSd
g. Mean walking step velocity for each day of active recording: WsV=ΣSd/ΣT (step/min)
h. Step length and total distance walked per day derived through biomechanical modelling
i. Variables a-h by time of the day
   (4) A sensor-based (e.g., accelerometer, gyroscope, magnetometer) and computer-implemented test for measures of orthostatic posture and balance, in particular, the Static Balance Test (SBT).

In one embodiment, the mobile device is adapted for performing or acquiring data from the Static Balance Test (SBT). The aim of this test is to assess a subject's static balance function as in one of the items (i.e., standing unsupported) of the widely used Berg Balance Scale (BBS), which is a 14-item objective measure designed to assess static balance and fall risk in adult populations. Data will be captured from smartphone and smartwatch sensors. The subjects are asked to stand still unsupported for 30 seconds with relaxed arms straight alongside the body if possible and with the smartphone in his/her pocket. Individuals with increased risk of falling and/or impaired static balance function may demonstrate altered postural control [sway] and abnormal arm movements. The test is typically performed daily.

Typical SBT performance parameters of interest:
1. Sway jerkiness: time derivative of acceleration (Mancini M et al. J Neuroeng Rehabil. 2012; 22: 9:59)
2. Sway path: total length of trajectory
3. Sway range
   (5) A computer-implemented test evaluating emotional status and well-being, in particular, the Mood Scale Question (MSQ).

In an embodiment, the mobile device is adapted for performing or acquiring data from a Mood Scale Question (MSQ) Questionnaire. Depression in its various forms is a common symptom of MS patients and if left untreated, it reduces quality of life, makes other symptoms—including fatigue, pain, cognitive changes—feeling worse, and may be life-threatening (National MS Society). Therefore in order to assess patients' perceived overall state, they will be asked how they feel through a 5-item questionnaire on the mobile device. The questionnaire is typically performed daily.

Typical MSQ performance parameters of interest:
1. Proportion of days with excellent mood in the last week, month, and year.
2. Proportion of days with≥good mood in the last week, month, and year.
3. Proportion of days with≥decent mood in the last week, month, and year.
4. Proportion of days with horrible mood in the last week, month, and year.
5. Frequency distribution of response type by time of the day between 6-8 am, 8-10 am, 10-12 am, 12-14, 14-16, 16-18, 18-20, 20-24, 0-6 am during the last month, and during the last year.
   (6) A computer-implemented test evaluating quality of life, in particular, the 29-Item Multiple Sclerosis Impact Scale (MSIS29).

In one embodiment, the mobile device is adapted for performing or acquiring data from the Multiple Sclerosis Impact Scale (MSIS)-29 test. To assess the impact of MS on the daily life of subjects, they will be asked to complete MSIS-29 (Hobart 2001, Brain 124: 962-73) biweekly on the mobile device, which is a 29-item questionnaire designed to measure the physical (items 1-20) and psychological (items 21-29) impact of MS from the patient's perspective (Hobart 2001, loc. cit.). We will use the second version of MSIS-29 (MSIS-29v2), which has four-point response categories for each item: “not at all,” “a little,” “moderately,” and “extremely”. MSIS-29 scores range from 29 to 116. Scores on the physical impact scale can range from 20 to 80 and on the psychological impact scale from 9 to 36, with lower scores indicating little impact of MS and higher scores indicating greater impact. Questions 4 and 5, as well as questions 2, 6, and 15 of MSIS-29 v.2 related to ambulation/lower limb and hand/arm/upper limb physical functions, respectively will also be subject to separate cluster analysis. The test is performed, typically, bi-weekly.

Typical MSIS-29 (v2) performance parameters of interest:
1. MSIS-29 score (29-116)
2. MSIS-29 Physical Impact Score (20-80)
3. MSIS-29 Psychological Impact Score (9-36)
4. MSIS-29 ambulation/lower limb score (2-8)
5. MSIS-29 hand/arm/upper limb score (3-12)
6. Time-corrected/filtered MSIS-29 scores of (1)-(5) based on minimum time needed to comprehend a posed question and provide an answer
7. Certainty weighted MSIS-29 scores of (1)-(6) based on the number of changes of a given answer and the difference/variation between the answers provided
8. Fine finger motor skill function parameters captured during MSIS-29
   a. Continuous variable analysis of duration of touchscreen contacts (Tts)
   b. Continuous variable analysis of deviation between touchscreen contacts (Dts) and center of closest target digit key
   c. Number of mistyped touchscreen contacts (Mts) (sum of contacts not triggering key hit or triggering key hit but associated with secondary sliding on screen), while typing responses
9. Ratio of 8a, 8b, and 8c variables versus corresponding variables of eSDMT (transformation/normalization of 8c to represent the projected number of Mts if MSIS-29 per 90 seconds)
   (7) A computer-implemented test tracking emerging new or worsening disease symptoms, in particular, the Multiple Sclerosis Symptom Tracker (MSST).

In yet an embodiment, the mobile device is adapted for performing or acquiring data from the Multiple Sclerosis Symptom Tracker (MSST). As the patient's perception of relapse occurrence and symptom variations may differ from clinically relevant symptom aggravation considered as a relapse, simple questions geared towards detecting new/worsening symptoms will be asked directly to the patients bi-weekly on the smartphone and synchronized with the MSIS-29 questionnaire. The patient has, in addition, the possibility to report symptoms and their respective calendar date of onset at any time. The MSST may, typically, be performed bi-weekly or on demand.

Typical MSST performance parameters of interest:
1. Number of reported episodes of “new or significantly worsening symptoms during the last two weeks” within the last month, and year (as per symptom onset date)
2. Proportion of total reported episodes of “new or significantly worsening symptoms during the last two weeks” that were considered to be “relapse(s)” vs. “not a relapse” vs. “unsure” within the last year
   (8) A computer-implemented passive monitoring of all or a predetermined subset of activities of a subject performed during a certain time window.

In yet another embodiment, the mobile device is adapted for performing or acquiring data from passive monitoring of all or a subset of activities. In particular, the passive monitoring shall encompass monitoring one or more activities performed during a predefined window, such as one or more days or one or more weeks, selected from the group consisting of: measurements of gait, the amount of movement in daily routines in general, the types of movement in daily routines, general mobility in daily living and changes in moving behavior.

Typical passive monitoring performance parameters of interest:
a. frequency and/or velocity of walking;
b. amount, ability and/or velocity to stand up/sit down, stand still and balance;
c. number of visited locations as an indicator of general mobility;
d. types of locations visited as an indicator of moving behavior.

It will be understood that the mobile device to be applied in accordance with this disclosure may be adapted to perform one or more of the aforementioned activity tests. In particular, it may be adapted to perform one, two, three, four, five, six, seven or all eight of these tests. Typically, combinations of tests may be implemented on the mobile device. Said combinations, more typically, comprise any one or all of test numbers (1) to (2). More particular, at least a test for fine motoric assessment as specified as test number (2) shall be implemented on the mobile device and, most typical, the Draw a Shape test and/or the Squeeze a Shape test.

Moreover, the mobile device may be adapted to perform further cognition and movement disorder and disease tests such as computer-implemented versions of other cognitive tests and/or the visual contrast acuity tests (such as low contrast letter acuity or Ishihara test; Ishihara test (see, e.g., Bove 2015, loc. cit.).

Further data may be processed in the method of this disclosure as well. These further data are typically suitable for further strengthening the identification of progressing MS in a subject. Typically, such data may be parameters from biochemical biomarkers for MS or data from imaging methods such as cross-sectional and/or longitudinal Magnetic Resonance Imaging (MRI) measures of whole brain volume, brain parenchymal fraction, whole grey matter volume, cortical grey matter volume, volume of specific cortical areas, deep grey matter volume, thalamic volume, corpus callosum surface or thickness, white matter volume, third ventricle volume, total brain T2-weighted hyperintense lesion volume, total cortical lesion volume, total brain T1-weighted hypointense lesion volume, total brain FLAIR (Fluid Attenuation Inversion Recovery) lesion volume, total new and/or enlarging T2 and FLAIR lesion number and volume, as assessed using automated algorithmic solution software, such as but not exclusively MSmetrix™, or NeuroQuant™.

The term “mobile device” as used herein refers to any portable device which comprises a sensor and data-recording equipment suitable for obtaining the dataset of activity measurements. Typically, the mobile device comprises a sensor for measuring the activity. This may also require a data processor and storage unit as well as a display for electronically simulating an activity test on the mobile device. Moreover, from the activity of the subject, data shall be recorded and compiled to a dataset which is to be evaluated by the method of the present disclosure either on the mobile device itself or on a second device. Depending on the specific setup envisaged, it may be necessary that the mobile device comprises data transmission equipment in order to transfer the acquired dataset from the mobile device to one or more further devices. Particularly well-suited mobile devices according to this disclosure are smartphones, smartwatches, wearable sensors, portable multimedia devices or tablet computers. Alternatively, portable sensors with data recording and, optionally, processing equipment may be used. Further, depending on the kind of activity test to be performed, the mobile device shall be adapted to display instructions for the subject regarding the activity to be carried out for the test. Particular envisaged activities to be carried out by the subject are described elsewhere herein and encompass the following tests: eSDMT, 2-Minute Walking Test (2MWT), 5 U-Turn Test (5UTT), Static balance test (SBT), Continuous Analysis of Gait (CAG), Draw a Shape, Squeeze a Shape, visual contrast acuity tests (such as low contrast letter acuity or Ishihara test), as well as other tests described in this specification.

Determining at least one parameter and, in particular, a fine motoric activity parameter or performance parameter as referred to herein, can be achieved by deriving a desired measured value from the dataset as the said parameter directly. Alternatively, the parameter may integrate one or more measured values from the dataset and, thus, may be derived from the dataset by mathematical operations. Typically, the parameter is derived from the dataset by an automated algorithm, e.g., by a computer program which automatically derives the said parameter from the dataset of activity measurements when tangibly embedded on a data processing device fed by the said dataset.

The term “reference” as used herein refers to a discriminator which allows the identification of a subject with a cognition and movement disease or disorder. Such a discriminator may be a value for the parameter which is indicative for subjects with cognition and movement disorders or diseases.

Such a value may be derived from one or more parameters, in particular, a fine motoric activity parameter or performance parameter as referred to herein, of subjects known to suffer from the cognition and movement disease or disorder to be investigated. Typically, the average or median may be used as a discriminator in such a case. If the determined parameter from the subject is identical to the reference or above a threshold derived from the reference, the subject can be identified as suffering from a cognition and movement disease or disorder in such a case. If the determined parameter differs from the reference and, in particular, is below the said threshold, the subject may be identified as not suffering from the cognition and movement disease or disorder, respectively.

Similarly, a value may be derived from one or more parameters, in particular, a fine motoric activity parameter or performance parameter as referred to herein, of subjects known not to suffer from a cognition and movement disease or disorder to be investigated. Typically, the average or median may be used as a discriminator in such a case. If the determined parameter from the subject is identical to the reference or below a threshold derived from the reference, the subject can be identified as not suffering from a cognition and movement disease or disorder in such a case. If the determined parameter differs from the reference and, in particular, is above the said threshold, the subject shall be identified as suffering from the cognition and movement disease or disorder.

As an alternative, the reference may be a previously determined parameter, in particular, a fine motoric activity parameter or performance parameter as referred to herein, from a dataset of activity measurements which has been obtained from the same subject prior to the actual dataset. In such a case, a parameter determined from the actual dataset which differs with respect to the previously determined parameter shall be indicative of either an improvement or worsening depending on the previous status of the disease and the kind of activity represented by the parameter. The skilled person knows based on the kind of activity and previous parameter how the said parameter can be used as a reference.

Comparing the determined at least one parameter, in particular, a fine motoric activity parameter or performance parameter as referred to herein, to a reference can be achieved by an automated comparison algorithm implemented on a data processing device such as a computer. The values of a determined parameter and a reference for said determined parameter are compared to each other as specified elsewhere herein in detail. As a result of the comparison, it can be assessed whether the determined parameter is identical, similar, or differs from or is in a certain relation to the reference (e.g., is larger or lower than the reference). Based on said assessment, the subject can be identified as suffering from a cognition and movement disease or disorder (“rule-in”), or not (“rule-out”). For the assessment, the kind of reference will be taken into account as described elsewhere in connection with suitable references according to this disclosure.

Moreover, by determining the degree of difference between a determined parameter and a reference, a quantitative assessment of a cognition and movement disease or disorder in a subject shall be possible. It is to be understood that an improvement, worsening or unchanged overall disease condition or of symptoms thereof can be determined by comparing an actually determined parameter to an earlier determined one used as a reference. Based on quantitative differences in the value of the said performance parameter the improvement, worsening or unchanged condition can be determined and, optionally, also quantified. If other references, such as references from subjects suffering from the cognition and movement disease or disorder to be investigated are used, it will be understood that the quantitative differences are meaningful if a certain disease stage can be allocated to the reference collective. Relative to this disease stage, worsening, improvement or unchanged disease condition can be determined in such a case and, optionally, also quantified.

The said diagnosis, i.e., the identification of the subject as being a subject suffering from a cognition and movement disease or disorder, or not, is indicated to the subject or other person, such as a medical practitioner. Typically, this is achieved by displaying the diagnosis on a display of the mobile device or the evaluation device. Alternatively, a recommendation for a therapy, such as a drug treatment, or for a certain life style, e.g., a certain nutritional diet or rehabilitation measures, is provided automatically to the subject or other person. To this end, the established diagnosis is compared to recommendations allocated to different diagnosis in a database. Once the established diagnosis matches one of the stored and allocated diagnoses, a suitable recommendation can be identified due to the allocation of the recommendation to the stored diagnosis matching the established diagnosis. Accordingly, it is, typically, envisaged that the recommendations and diagnoses are present in form of a relational database. However, other arrangements which allow for the identification of suitable recommendations are also possible and known to the skilled artisan.

Moreover, the one or more parameter may also be stored on the mobile device or indicated to the subject, typically, in real time. The stored parameters may be assembled into a time course or similar evaluation measures. Such evaluated parameters may be provided to the subject as a feedback for activity capabilities investigated in accordance with the method of this disclosure. Typically, such a feedback can be provided in electronic format on a suitable display of the mobile device and can be linked to a recommendation for a therapy as specified above or rehabilitation measures.

Further, the evaluated parameters may also be provided to medical practitioners in doctors' offices or hospitals as well as to other health care providers, such as, developers of diagnostic tests or drug developers in the context of clinical trials, health insurance providers or other stakeholders of the public or private health care system.

Typically, the method of the present disclosure for assessing a subject suffering from a cognition and movement disease or disorder may be carried out as follows:

First, at least one cognition and/or fine motoric activity parameter is determined from an existing dataset of activity measurements obtained from said subject using a mobile device. Said dataset may be transmitted from the mobile device to an evaluating device, such as a computer, or may be processed in the mobile device in order to derive the said at least one parameter from the dataset.

Second, the determined at least one cognition and/or fine motoric activity parameter is compared to a reference by, e.g., using a computer-implemented comparison algorithm carried out by the data processor of the mobile device or by the evaluating device, e.g., the computer. The result of the comparison is assessed with respect to the reference used in the comparison and based on the said assessment the subject will be assessed with respect to the cognition and movement disease or disorder.

Third, the said assessment, e.g., the identification of the subject as being a subject suffering from the cognition and movement disease or disorder, or not, is indicated to the subject or other person, such as a medical practitioner.

Alternatively, a recommendation for a therapy, such as a drug treatment, or for a certain life style, e.g., a certain nutritional diet, is provided automatically to the subject or other person. To this end, the established assessment is compared to recommendations allocated to different assessments in a database. Once the established assessment matches one of the stored and allocated assessments, a suitable recommendation can be identified due to the allocation of the recommendation to the stored assessment matching the established assessment. Typical recommendations involve therapeutic measures as described elsewhere herein.

Yet as an alternative or in addition, the at least one parameter underlying the assessment will be stored on the mobile device. Typically, it shall be evaluated together with other stored parameters by suitable evaluation tools, such as time course assembling algorithms, implemented on the mobile device which can assist electronically with rehabilitation or therapy recommendations as specified elsewhere herein.

This disclosure, in light of the above, also specifically contemplates a method of assessing a cognition and movement disease or disorder in a subject comprising the steps of:
a) obtaining from said subject using a mobile device a dataset of cognition and/or fine motoric activity measurements during predetermined activity performed by the subject;
b) determining at least one cognition and/or fine motoric activity parameter determined from a dataset of activity measurements obtained from said subject using a mobile device;
c) comparing the determined at least one cognition and/or fine motoric activity parameter to a reference; and
d) assessing the cognition and movement disease or disorder in a subject based on the comparison carried out in step (c).

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it should be understood that all terms used throughout this disclosure and claims, regardless of whether said terms are preceded by the phrases “one or more, “at least one, or the like, should not receive a singular interpretation unless it is made explicit herein. That is, all terms used in this disclosure and claims should generally be interpreted to mean “one or more” or “at least one.”

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, notwithstanding the fact that the respective feature or element may be present once or more than once.

Further, as used in the following, the terms “particularly,” “more particularly,” “specifically,” “more specifically,” “typically,” and “more typically” or similar terms are used in conjunction with additional/alternative features, without restricting alternative possibilities. Thus, features introduced by these terms are additional/alternative features and are not intended to restrict the scope of the claims in any way. The invention may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative features of the invention.

Advantageously, it has been found in the studies underlying this disclosure that fine motoric activity parameters, optionally together with other performance parameters of motoric and cognitive capabilities, obtained from datasets measured during certain activities of patients suspected to suffer or suffering from a cognition and movement disease or disorder can be used as digital biomarkers for assessing, e.g., identifying or monitoring, those patients which suffer from the said disorder or disease. The said datasets can be acquired from the patients in a convenient manner by using mobile devices such as the omnipresent smart phones, portable multimedia devices or tablet computers. The datasets thereby acquired can be subsequently evaluated by the method of the invention for the at least one cognition or fine motoric activity parameter suitable as digital biomarker. Said evaluation can be carried out on the same mobile device or it can be carried out on a separate remote device. Moreover, by using such mobile devices, recommendations on life style or therapy can be provided to the patients directly, i.e., without the consultation of a medical practitioner in a doctor's office or hospital. Thanks to the present invention, the life conditions of patients can be adjusted more precisely to the actual disease status due to the use of actual determined parameters by the method of the disclosure. Thereby, drug treatments can be selected that are more efficient or dosage regimens can be adapted to the current status of the patient. It is to be understood that the disclosed method is, typically, a data evaluation method which requires an existing dataset of cognition or fine motoric activity measurements from a subject. Within this dataset, the method determines at least one cognition or fine motoric activity parameter which can be used for assessing a cognition and movement disease or disorder, i.e., which can be used as a digital biomarker for said disease or disorder.

Accordingly, the method of the present disclosure may be used for:
assessing the disease condition;
monitoring patients, in particular, in a real life, daily situation and on large scale;
supporting patients with life style and/or therapy recommendations;
investigating drug efficacy, e.g., also during clinical trials;
facilitating and/or aiding therapeutic decision making;
supporting hospital management;
supporting rehabilitation measure management;
supporting health insurance assessments and management; and/or
supporting decisions in public health management.

The explanations and definitions for the terms made above apply mutatis mutandis to the embodiments described herein below.

In the following, particular embodiments of the method of the present invention are described:

In an embodiment of the method of the present disclosure, said cognition and movement disease or disorder is a disease or disorder of the central and/or peripheral nervous system affecting the pyramidal, extrapyramidal, sensory or cerebellar system, or a neuromuscular disease or is a muscular disease or disorder.

In yet an embodiment of the method of this disclosure, said cognition and movement disease or disorder is selected from the group consisting of: multiple sclerosis, stroke, a cerebellar disorder, cerebellar ataxia, spastic paraplegia, essential tremor, myasthemia or other forms of neuromuscular disorders, muscular dystrophy, myositis or other muscular disorders, a peripheral neuropathy, cerebral palsy, extrapyramidal syndromes, Alzheimers disease, other forms of dementia, leukodystrophies, autism spectrum disorders, attention-deficit disorders (ADD/ADHD), intellectual disabilities as defined by DSM-5, impairment of cognitive performance and reserve related to aging, Parkinson's disease, Huntington's disease, a polyneuropathy, and amyotrophic lateral sclerosis.

In particular, it has been found that the subjects suffering from NMO and NMOSD, cerebellar ataxia, spastic paraplegia, essential tremor, myasthemia or other forms of neuromuscular disorders, muscular dystrophy, myositis or other muscular disorders, a peripheral neuropathy may be identified efficiently by using fine motoric activity datasets obtained from the draw a shape and/or squeeze a shape tests. Subjects suffering from cerebral palsy, extrapyramidal syndromes, Alzheimer's disease, other forms of dementia, leukodystrophies, autism spectrum disorders, attention-deficit disorders (ADD/ADHD), intellectual disabilities as defined by DSM-5, impairment of cognitive performances and reserve related to aging may be identified efficiently from fine motoric activity datasets obtained from the eSDMT test. The remaining disease or disorders may be identified by fine motoric activity datasets from any tests or from the combination of all tests efficiently. Accordingly, depending on the cognition and movement disease or disorder to be investigated, the mobile device may be individually configured for obtaining datasets from a suitable combination of tests.

In an embodiment of the method of this disclosure, the at least one cognition and/or fine motoric activity parameter is a parameter being indicative for attention, information procession speed, and/or hand motoric functions.

In another embodiment of the method of this disclosure, the said dataset of fine motoric activity measurements comprises data from a test encompassing drawing shapes with a finger (Draw a shape test) and/or squeezing shapes with a finger (squeeze a shape test) on a sensor surface of the mobile device.

In an embodiment of the method of this disclosure, said dataset of cognition activity measurements comprises data from a test encompassing performing a eSDMT test on a sensor surface of the mobile device.

In yet another embodiment of the method of this disclosure, in addition, at least one performance parameter from a dataset of activity measurements is determined as being indicative for the subject's other motoric capabilities and function, walking, color vision, attention, dexterity and/or cognitive capabilities, quality of life, fatigue, mental state, mood, vision and/or cognition.

In a further embodiment of the method of this disclosure, in addition at least one performance parameter from a dataset of activity measurements is determined selected from the group consisting of: 2-Minute Walking Test (2MWT), 5 U-Turn Test (5UTT), Static balance test (SBT), Continuous Analysis of Gait (CAG), visual contrast acuity tests (such as low contrast letter acuity or Ishihara test), Mood Scale Question (MSQ), MSIS-29, and passive monitoring of all or a predetermined subset of activities of a subject performed during a certain time window.

In an embodiment of the method of this disclosure, said mobile device has been adapted for carrying out on the subject one or more of the tests referred to above for cognition and/or fine motoric activity measurements and, preferably, also tests for determining at least one performance parameter.

In an embodiment of the method of this disclosure, said mobile device is embodied as a smartphone, smartwatch, wearable sensor, portable multimedia device or tablet computer.

In a further embodiment of the method of this disclosure, said reference is at least one cognition and/or fine motoric activity parameter derived from a dataset of cognition and/or fine motoric activity measurements obtained from the said subject at a time point prior to the time point when the dataset of cognition and/or fine motoric activity measurements referred to in step a) has been obtained from the subject. Typically, a worsening between the determined at least one cognition and/or fine motoric activity parameter and the reference is indicative for a subject that suffers from the cognition and movement disease or disorder.

In another embodiment of the method of this disclosure, said reference is at least one at least one cognition and/or fine motoric activity parameter derived from a dataset of cognition and/or fine motoric activity measurements obtained from a subject or group of subjects known to suffer from the cognition and movement disease or disorder. Typically, a determined at least one cognition and/or fine motoric activity parameter being essentially identical compared to the reference is indicative of a subject that suffers from the cognition and movement disease or disorder.

In a further embodiment of the method of this disclosure, said reference is at least one cognition and/or fine motoric activity parameter derived from a dataset of cognition and/or fine motoric activity measurements obtained from a subject or group of subjects known not to suffer from the cognition and movement disease or disorder. Typically, a determined at least one cognition and/or fine motoric activity parameter being worsened compared to the reference is indicative of a subject that suffers from the cognition and movement disease or disorder.

This disclosure also contemplates a computer program, computer program product or computer readable storage medium having tangibly embedded said computer program, wherein the computer program comprises instructions that, when run on a data processing device or computer, carry out the method of the present disclosure as specified above. Specifically, the present disclosure further encompasses:
A computer or computer network comprising at least one processor, wherein the processor is adapted to perform the method according to one of the embodiments described;
a computer loadable data structure that is adapted to perform the method according to one of the described embodiments while the data structure is being executed on a computer;
a computer script, wherein the computer program is adapted to perform the method according to one of the embodiments described in this description while the program is being executed on a computer;
a computer program comprising program means for performing the method according to one of the embodiments described in this description while the computer program is being executed on a computer or on a computer network;
a computer program comprising program means according to the preceding embodiment, wherein the program means are stored on a storage medium readable to a computer;
a storage medium, wherein a data structure is stored on the storage medium and wherein the data structure is adapted to perform the method according to one of the embodiments described in this description after having been loaded into a main and/or working storage of a computer or of a computer network;
a computer program product having program code means, wherein the program code means can be stored or are stored on a storage medium, for performing the method according to one of the embodiments described in this description, if the program code means are executed on a computer or on a computer network;
a data stream signal, typically encrypted, comprising a dataset of cognition or fine motoric activity measurements obtained from the subject using a mobile device; and
a data stream signal, typically encrypted, comprising the at least one cognition or fine motoric activity parameter derived from the dataset of cognition or fine motoric activity measurements obtained from the subject using a mobile device.

This disclosure, further, relates to a method for determining at least one cognition or fine motoric activity parameter from a dataset of cognition or fine motoric activity measurements obtained from said subject using a mobile device
a) deriving at least one cognition or fine motoric activity parameter from a dataset of cognition or fine motoric activity measurements obtained from said subject using a mobile device; and
b) comparing the determined at least one cognition or fine motoric activity parameter to a reference,
   wherein, typically, said at least one cognition or fine motoric activity parameter can aid assessing a cognition and movement disease or disorder in said subject.

This disclosure also relates to a method for recommending a therapy for a cognition and movement disease or disorder comprising the steps of the aforementioned method of the disclosure (i.e., the method for identifying a subject as suffering from a cognition and movement disease or disorder) and the further step of recommending the therapy if the cognition and movement disease or disorder is assessed.

The term “a therapy for a cognition and movement disease or disorder” as used herein refers to all kinds of medical treatments, including drug-based therapies, surgeries, psychotherapy, physical-therapy and the like. The term also encompasses life-style recommendations, rehabilitation measures, and recommendations of nutritional diets. Typically, the method encompasses recommendation of a drug-based therapy and, in particular, a therapy with a drug known to be useful for the treatment of the cognition and movement disease or disorder. Such drugs may be a therapy with one or more drugs selected from the group consisting of: interferon beta-1a, interferon beta-1b, glatiramer acetate, mitoxantrone, natalizumab, fingolimod, teriflunomide, dimethyl fumarate, alemtuzumab, daclizumab, thrombolytic agents, such as recombinant tissue plasmin activator, acetylcholinesterase inhibitors, such as tacrine, rivastigmine, galantamine or donepezil, NMDA receptor antagonists, such as memantine, non-steroidal anti-inflammatory drugs, dopa carboxylase inhibitors, such as levodopa, tolcapone or entacapone, dopamine antagonists, such as bromocriptine, pergolide, pramipexole, ropinirole, piribedil, cabergoline, apomorphine or lisuride, MAO-B inhibitors, such as safinamide, selegiline or rasagiline, amantadine, anticholinergics, tetrabenazine, neuroleptics, benzodiazepines, and riluzole. Moreover, the aforementioned method may comprise in yet an embodiment the additional step of applying the recommended therapy to the subject.

Moreover, encompassed in accordance with this disclosure is a method for determining efficacy of a therapy against a cognition and movement disease or disorder comprising the steps of the aforementioned method of this disclosure (i.e., the method for identifying a subject as suffering from a cognition and movement disease or disorder) and the further step of determining a therapy response if improvement of the cognition and movement disease or disorder occurs in the subject upon therapy or determining a failure of response if worsening of the cognition and movement disease or disorder occurs in the subject upon therapy or if the cognition and movement disease or disorder remains unchanged.

The term “improvement” as referred to in accordance with this disclosure relates to any improvement of the overall disease or disorder condition or of individual symptoms thereof. Likewise, a “worsening” means any worsening of the overall disease or disorder condition or individual symptoms thereof. Since the course of some cognition and movement disorders may be associated typically with a worsening of the overall disease or disorder condition and symptoms thereof, the worsening referred to in connection with the aforementioned method is an unexpected or untypical worsening which goes beyond the normal course of disease or disorder progression. Unchanged in this context may, thus, also mean that the overall disease or disorder condition and the symptoms accompanying it are within the normal cause of disease or disorder progression.

Further, this disclosure contemplates a method of monitoring a cognition and movement disease or disorder in a subject comprising determining whether the cognition and movement disease or disorder improves, worsens or remains unchanged in a subject by carrying out the steps of the aforementioned method of this disclosure (i.e., the method for identifying a subject as suffering from a cognition and movement disease or disorder) at least two times during a predefined monitoring period.

The term “predefined monitoring period” as used herein refers to a predefined time period in which at least two times activity measurements are carried out. Typically, such a period may range from days to weeks to months to years depending on the course of disease or disorder progression to be expected for the individual subject. Within the monitoring period, the activity measurements and parameters are determined at a first time point which is usually the start of the monitoring period and at least one further time point. However, it is also possible that there are more than one further time points for activity measurements and parameter determination. In any event, the fine motoric activity parameter(s) determined from the activity measurements of the first time point are compared to such parameters of subsequent time points. Based on such a comparison, quantitative differences can be identified which will be used to determine a worsening, improved or unchanged disease condition during the predefined monitoring period.

This disclosure relates to a mobile device comprising a processor, at least one sensor and a database as well as software which is tangibly embedded in said device and, when running on said device, carries out any one of the methods of this disclosure.

Further contemplated is a system comprising a mobile device comprising at least one sensor and a remote device comprising a processor and a database as well as software which is tangibly embedded in said device and, when running on said device, carries out any one of the methods of this disclosure, wherein said mobile device and said remote device are operatively linked to each other.

Under “operatively linked to each other” it is to be understood that the devices are connect as to allow data transfer from one device to the other device. Typically, it is envisaged that at least the mobile device which acquires data from the subject is connected to the remote device carrying out the steps of the disclosed methods such that the acquired data can be transmitted for processing to the remote device. However, the remote device may also transmit data to the mobile device such as signals controlling or supervising its proper function. The connection between the mobile device and the remote device may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Further details may be found elsewhere in this specification. For data acquisition, the mobile device may comprise a user interface such as screen or other equipment for data acquisition. Typically, the activity measurements can be performed on a screen comprised by a mobile device, wherein it will be understood that the said screen may have different sizes including, e.g., a 5.1 inch screen.

Also, this disclosure relates to the use of the mobile device or the system of the present disclosure for identifying a subject suffering from a cognition and movement disease or disorder.

The present disclosure also contemplates the use of the mobile device or the system according to this disclosure for monitoring a subject suffering from a cognition and movement disease or disorder, in particular, in a real life, daily situation and on large scale.

Yet, it will be understood that this disclosure contemplates the use of the mobile device or the system according to the present invention for investigating drug efficacy, e.g., also during clinical trials, in a subject suffering from a cognition and movement disease or disorder.

Further, this disclosure contemplates the use of the mobile device or the system according to the present disclosure for facilitating and/or aiding therapeutic decision making for a subject suffering from a cognition and movement disease or disorder.

Furthermore, this disclosure provides for the use of the mobile device or the system according to the present disclosure for supporting hospital management, rehabilitation measure management, health insurance assessments and management and/or supporting decisions in public health management with respect to subjects suffering from a cognition and movement disease or disorder.

Encompassed by this disclosure is furthermore the use of the mobile device or the system according to the present disclosure for supporting a subject suffering from a cognition and movement disease or disorder with life style and/or therapy recommendations.

Further particular embodiments are also listed as follows:

Embodiment 1: A method for assessing a cognition and movement disease or disorder in a subject suspected to suffer therefrom comprising the steps of:
a) determining at least one cognition and/or fine motoric activity parameter from a dataset of fine motoric activity measurements obtained from said subject using a mobile device; and
b) comparing the determined at least one cognition and/or fine motoric activity parameter to a reference, whereby the cognition and movement disease or disorder will be assessed.

Embodiment 2: The method of embodiment 1, wherein said cognition and movement disease or disorder is a disease or disorder of the central and/or peripheral nervous system affecting the pyramidal, extrapyramidal, sensory or cerebellar system, or a neuromuscular disease or is a muscular disease or disorder.

Embodiment 3: The method of embodiment 1 or 2, wherein said cognition and movement disease or disorder is selected from the group consisting of: multiple sclerosis (MS), neuromyelitis optica (NMO) and NMO spectrum disorders, stroke, a cerebellar disorder, cerebellar ataxia, spastic paraplegia, essential tremor, myasthenia and myasthenic syndromes or other forms of neuromuscular disorders, muscular dystrophy, myositis or other muscular disorders, a peripheral neuropathy, cerebral palsy, extrapyramidal syndromes, Parkinson's disease, Huntington's disease, Alzheimer's disease, other forms of dementia, leukodystrophies, autism spectrum disorders, attention-deficit disorders (ADD/ADHD), intellectual disabilities as defined by DSM-5, impairment of cognitive performance and reserve related to aging, Parkinson's disease, Huntington's disease, a polyneuropathy, motor neuron diseases and amyotrophic lateral sclerosis (ALS).

Embodiment 4: The method of any one of embodiments 1 to 3, wherein the at least one fine motoric activity parameter is indicative of hand motoric functions.

Embodiment 5: The method of any one of embodiments 1 to 4, wherein the said dataset of fine motoric activity measurements comprises data from a test encompassing drawing shapes with a finger (Draw a shape test) and/or squeezing shapes with a finger (Squeeze a shape test) on a sensor surface of the mobile device.

Embodiment 6: The method of any one of embodiments 1 to 5, wherein the said dataset of cognition activity measurements comprises data from a test encompassing performing a eSDMT test on a sensor surface of the mobile device.

Embodiment 7: The method of any one of embodiments 1 to 6, wherein in addition at least one performance parameter from a dataset of activity measurements is determined that is indicative of the subject's other motoric capabilities and function, walking, color vision, attention, dexterity and/or cognitive capabilities, quality of life, fatigue, mental state, mood, vision and/or cognition.

Embodiment 8: The method of any one of embodiments 1 to 7, wherein in addition at least one performance parameter from a dataset of activity measurements is determined and is selected from the group consisting of: 2-Minute Walking Test (2MWT), 5 U-Turn Test (5UTT), Static balance test (SBT), Continuous Analysis of Gait (CAG), visual contrast acuity tests (such as low contrast letter acuity or Ishihara test), Mood Scale Question (MSQ), MSIS-29, and passive monitoring of all or a predetermined subset of activities of a subject performed during a certain time window.

Embodiment 9: The method of any one of embodiments 1 to 8, wherein said mobile device has been adapted for carrying out on the subject one or more of the tests referred to in embodiment 4, 5 and/or 6 and, preferably, also embodiments 7 and/or 8.

Embodiment 10: The method of embodiment 9, wherein said mobile device is comprised in a smartphone, smartwatch, wearable sensor, portable multimedia device or tablet computer.

Embodiment 11: The method of any one of embodiments 1 to 10, wherein said reference is at least one cognition and/or fine motoric activity parameter derived from a dataset of cognition and/or fine motoric activity measurements obtained from the said subject at a time point prior to the time point when the dataset of cognition and/or fine motoric activity measurements referred to in step a) has been obtained from the subject.

Embodiment 12: The method of embodiment 11, wherein a worsening between the determined at least one cognition and/or fine motoric activity parameter and the reference is indicative of a subject that suffers from the cognition and movement disease or disorder.

Embodiment 13: The method of any one of embodiments 1 to 10, wherein said reference is at least one cognition and/or fine motoric activity parameter derived from a dataset of cognition and/or fine motoric activity measurements obtained from a subject or group of subjects known to suffer from the cognition and movement disease or disorder.

Embodiment 14: The method of embodiment 13, wherein a determined at least one cognition and/or fine motoric activity parameter being essentially identical compared to the reference is indicative of a subject that suffers from the cognition and movement disease or disorder.

Embodiment 15: The method of any one of embodiments 1 to 10, wherein said reference is at least one cognition and/or fine motoric activity parameter derived from a dataset of cognition and/or fine motoric activity measurements obtained from a subject or group of subjects known not to suffer from the cognition and movement disease or disorder.

Embodiment 16: The method of claim 15, wherein a determined at least one cognition and/or fine motoric activity parameter being worsened compared to the reference is indicative of a subject that suffers from the cognition and movement disease or disorder.

Embodiment 17: A method for recommending a therapy for a cognition and movement disease or disorder comprising the steps of the method of any one of embodiments 1 to 16 and the further step of recommending the therapy if the cognition and movement disease or disorder is assessed.

Embodiment 18: A method for determining efficacy of a therapy against a cognition and movement disease or disorder comprising the steps of the method of any one of embodiments 1 to 16 and the further step of determining a therapy response if improvement of the cognition and movement disease or disorder occurs in the subject upon therapy or determining a failure of response if worsening of the cognition and movement disease or disorder occurs in the subject upon therapy or if the cognition and movement disease or disorder remains unchanged.

Embodiment 19: A method of monitoring a cognition and movement disease or disorder in a subject comprising determining whether the cognition and movement disease or disorder improves, worsens or remains unchanged in a subject by carrying out the steps of the method of any one of embodiments 1 to 16 at least two times during a predefined monitoring period.

Embodiment 20: A mobile device comprising a processor, at least one sensor and a database as well as software which is tangibly embedded in said device and, when running on said device, carries out the method of any one of embodiments 1 to 19.

Embodiment 21: A system comprising a mobile device comprising at least one sensor and a remote device comprising a processor and a database as well as software which is tangibly embedded in said device and, when running on said device, carries out the method of any one of embodiments 1 to 19, wherein said mobile device and said remote device are operatively linked to each other.

Embodiment 22: A mobile device of the embodiment 20 or the system of embodiment 21 for use in identifying a subject suffering from a cognition and movement disease or disorder.

Embodiment 23: A mobile device of embodiment 20 or the system of embodiment 21 for use in monitoring a subject suffering from a cognition and movement disease or disorder, in particular, in a real life, daily situation and on large scale, for investigating drug efficacy, e.g., also during clinical trials, in a subject suffering from a cognition and movement disease or disorder, for facilitating and/or aiding therapeutic decision making for a subject suffering from a cognition and movement disease or disorder, for supporting hospital management, rehabilitation measure management, health insurance assessments and management and/or supporting decisions in public health management with respect to subjects suffering from a cognition and movement disease or disorder or for supporting a subject suffering from a cognition and movement disease or disorder with life style and/or therapy recommendations.

All references cited throughout this specification are herewith incorporated by reference with respect to their entire disclosure content and with respect to the specific disclosure contents mentioned in the specification.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIGS. 1A, 1B, 1C and 1D show a smartphone adapted for performing a computer implemented Draw a Shape test. FIG. 1A) Instructions are given to the patient on the screen of the smartphone; FIGS. 1B, 1C and 1D) user interfaces for testing drawing different shapes.

FIGS. 2A, 2B, 2C and 2D show a smartphone adapted for performing a computer implemented Squeeze a Shape test. FIG. 2A) Instructions are given to the patient on the screen of the smartphone; FIGS. 2B, 2C and 2D) user interface showing the different stages of a squeezing the shape activity.

FIGS. 3A, 3B and 3C show a smartphone adapted for performing a computer-implemented eSDMT. FIG. 3A) Instructions are given to the patient on the screen of the smartphone; FIG. 3B) user interface for testing matching digits; FIG. 3C) user interface for testing matching symbols.

FIGS. 4A and 4B show an eSDMT test performance of 30 subjects. FIG. 4A shows the distribution of number of total responses. The accuracy rate is depicted in 4B.

FIGS. 5A, 5B, 5C, 5D, 5E and 5F show the time elapsed between subsequent responses (R) and subsequent correct responses (CR) in eSDMT tests. FIGS. 5A, 5C and 5E show the elapsed time between subsequent responses (R). FIGS. 5B, 5D and 5F show the elapsed time between subsequent correct responses (CR). The subject population is divided into three groups: FIGS. 5A and 5B stem from subjects providing fewer than 32 (correct) responses (N=9); FIGS. 5C and 5D from subjects providing between 32 and 39 (correct) responses (N=10); and FIGS. 5E and 5F provide 40 or more (correct) responses (N=11) over the course of 90 seconds. The median of the elapsed time is plotted as line and the standard deviation is shown as shaded region.

FIGS. 6A, 6B, 6C and 6D show examples of responses (R) and correct responses (CR) profile of two subjects with quite distinct performances in eSDMT tests. FIG. 6A shows the cumulative responses (R) profile of two subjects over 90 seconds. FIG. 6C shows the elapsed time between subsequent responses (R) of two patients. FIG. 6B shows the cumulative correct responses (CR) profile of two patients over 90 seconds. FIG. 6D shows the elapsed time between subsequent correct responses (CR) of two patients.

FIGS. 7A, 7B, 7C, 7D and 7E show an illustration of Squeeze a Shape test data. FIG. 7A shows an overview of a subject performing the Squeeze a Shape test for 30 seconds. Circles 110 in FIG. 7B illustrate the touch events from the first finger and circles 112 show second finger touch. Circles 114 in FIG. 7B show whenever two contact points with the display were made at the same time. The vertical dotted lines show the start and end of a pinch attempt, respectively. Line 116 in FIG. 7C shows the distance between the two pinching fingers. FIG. 7D shows the speed of the first and second fingers. FIG. 7E shows the distance between the two pinching fingers.

FIGS. 8A and 8B show examples of touch traces for a circle shape from two subjects. Circles 120 along the dashed line indicate waypoints that subjects have to pass through. Circles 122 are the trace points. Each crosshair 118 represents the closest trace point 122 to each waypoint 120. FIG. 8A depicts a subject with poor 9HPT. FIG. 8B shows the baseline subject chosen based on good 9HPT performance.

FIGS. 9A, 9B and 9C show the tracing performance for examples shown in FIG. 8. Error distances per each waypoint of circle shape are shown in FIG. 9A. FIG. 9B shows shape specific segmentation into sectors, and subsequent error per sector. FIG. 9C shows the range of error distances per subject, including median and IQR.

FIGS. 10A and 10B show examples of touch traces for spiral shape from two subjects. Circles 120 along the dashed line indicate waypoints that subjects have to pass through. Circles 122 are the trace points. Each crosshair 118 represents the closest trace point 122 to each waypoint 120. FIG. 10A depicts a subject with poor 9HPT. FIG. 10B shows the baseline subject chosen based on good 9HPT performance.

FIGS. 11A, 11B and 11C show the tracing performance. Error distances per each waypoint of spiral shape are shown in FIG. 11A. FIG. 11B shows shape specific segmentation into sectors, and subsequent error per sector. FIG. 11C shows the range of error distances per subject, including median and IQR.

FIGS. 12A, 12B and 12C show the collective spatial and temporal characteristics of a subject's drawing performance through visual, velocity and acceleration analysis. Velocity is calculated as the change in Euclidean distance between consecutive points over time; acceleration is the rate of change of velocity over time. Through this shape and subject specific complementary analysis to a spatial analysis of points drawn, a subject's fine temporal performance characteristics can be studied. FIG. 12A) visual tracing of specified shape. FIG. 12B) velocity tracing of draw-a-shape task over time to complete [s]. FIG. 12C) acceleration tracing of Draw-a-Shape task over time to complete [s].

FIGS. 13A and 13B compare patient adherence to active tests and passive monitoring. Adherence count is based on adherent days per study week, defined as the week starting from the first data point received by the respective subject. Amount of passive monitoring collected is based on the duration of accelerometer recordings with correction for inactivity for smartphones and smartwatches individually. 2MWT, Two-Minute Walking Test.

FIG. 14 shows an association between PROs conducted on the smartphone and in the clinic. Total scores of paper-based MSIS-29 and smartphone-based MSIS-29 are compared at baseline (screening visit). The identity line is depicted as a dashed line. MSIS-29, Multiple Sclerosis Impact Scale.

FIG. 15 shows a cross-sectional baseline correlation of oral SDMT vs smartphone-based SDMT. At baseline, the number of correct responses from the smartphone-based SDMT correlated with correct responses from the oral SDMT (Spearman's correlation coefficient=0.72, p<0.001). The patient-level performances on oral SDMT were overall better than on the smartphone-based SDMT.

FIGS. 16A and 16B show that turning speed while walking correlates with T25FW (FIG. 16A) and EDSS (FIG. 16B). FIG. 16A shows turning speed measured with the 5UTT correlates with the T25FW (Spearman's correlation coefficient=−0.62, p<0.001); as well as the ambulation items (items 4 and 5) of the MSIS-29 (Spearman's correlation coefficient=−0.57, p=0.001). FIG. 16B shows turning speed measured with the 5UTT correlates with the EDSS score (Spearman's correlation coefficient=−0.72, p<0.001).

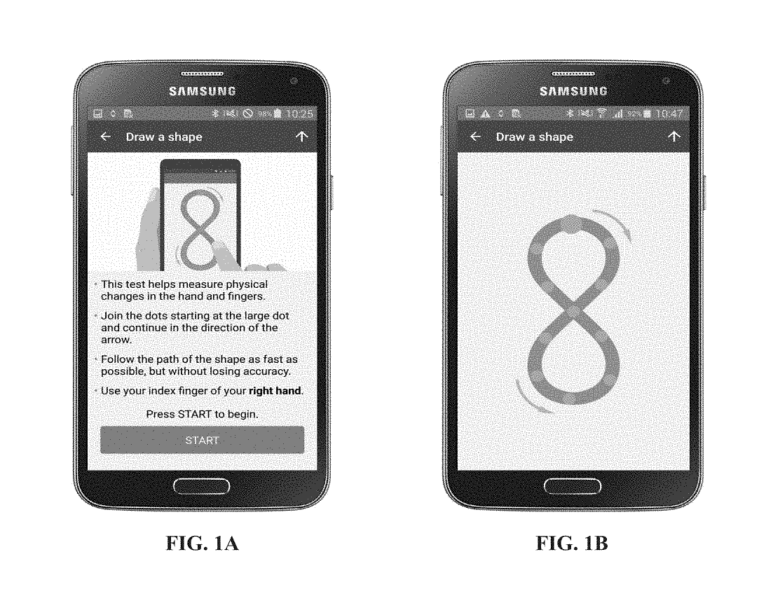

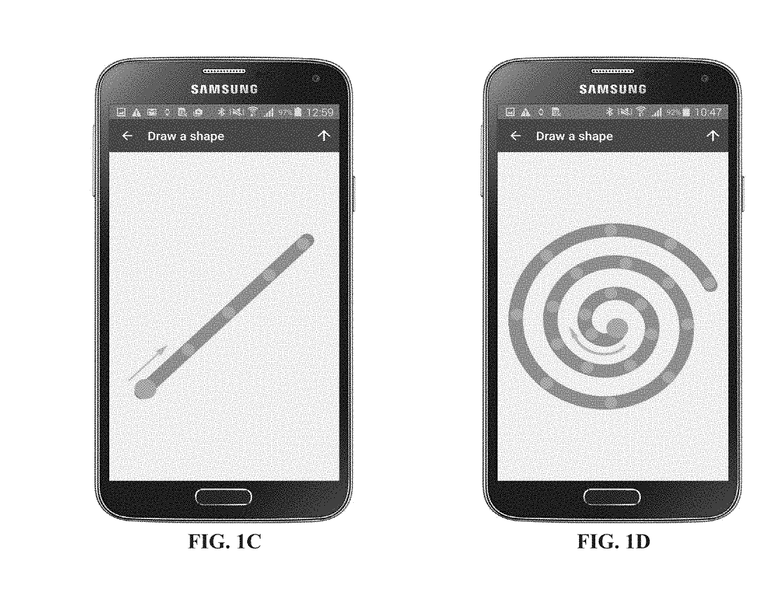

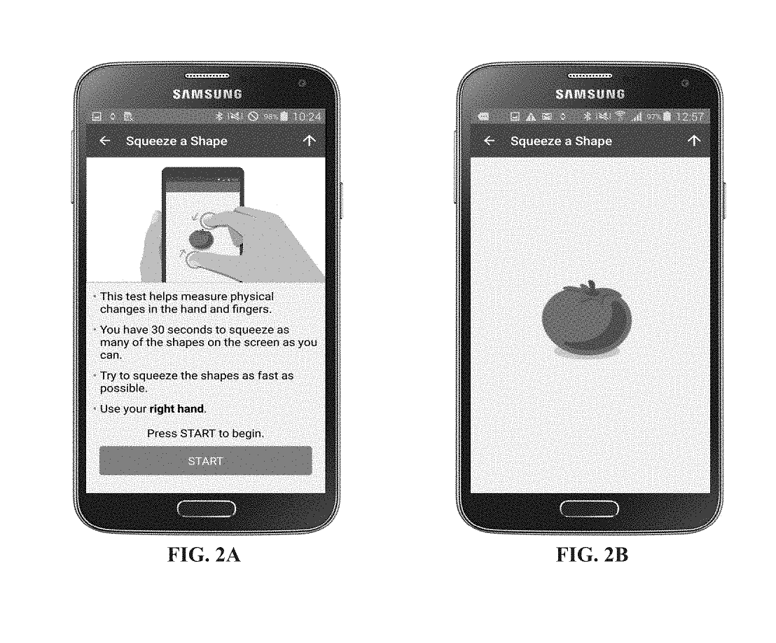

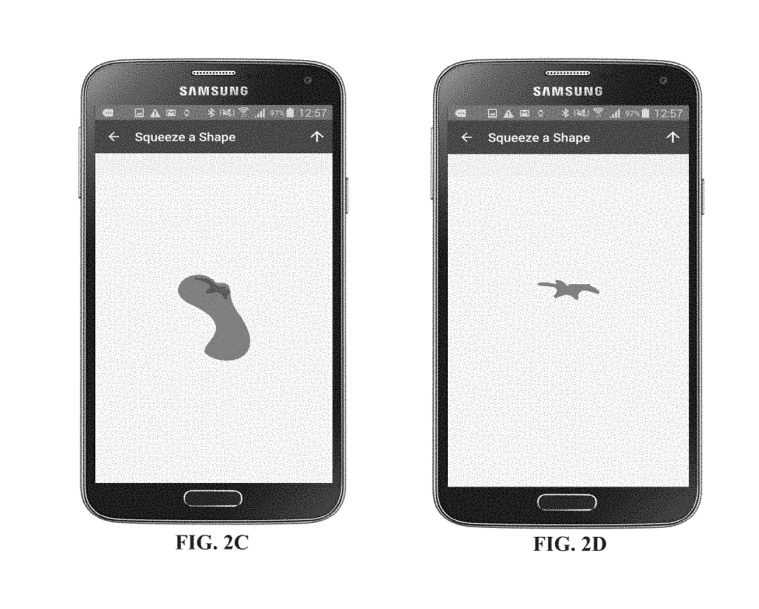

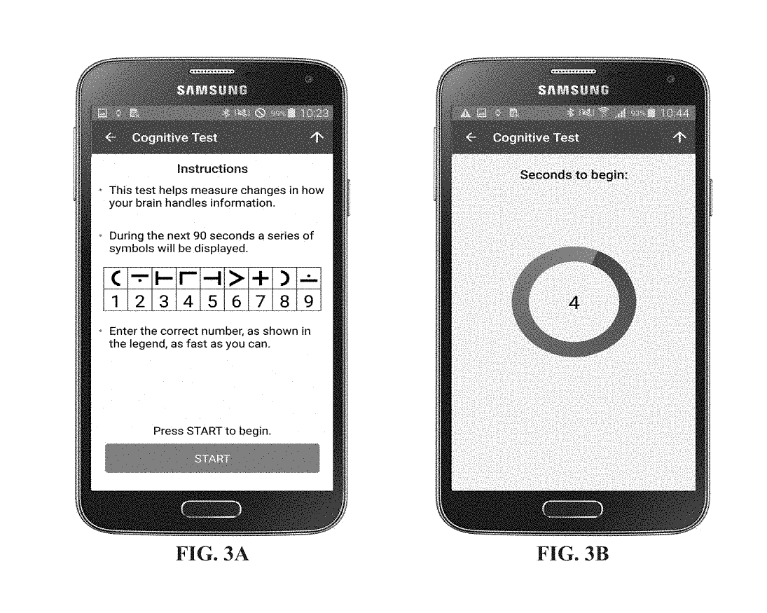

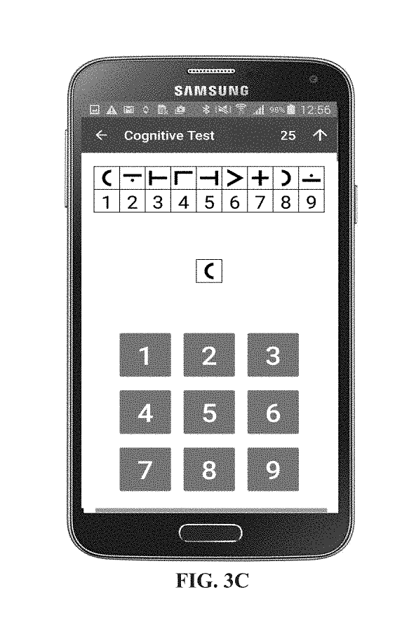

### DESCRIPTION AND EXAMPLES

The embodiments and examples described below are not intended to be exhaustive or to limit the invention to the precise forms disclosed in the following detailed description. Rather, the embodiments are chosen and described so that others skilled in the art may appreciate and understand the principles and practices of this disclosure.

### EXAMPLE 1

### A Computer-Implemented (Electronic) Symbol Digit Modalities Test (eSDMT)

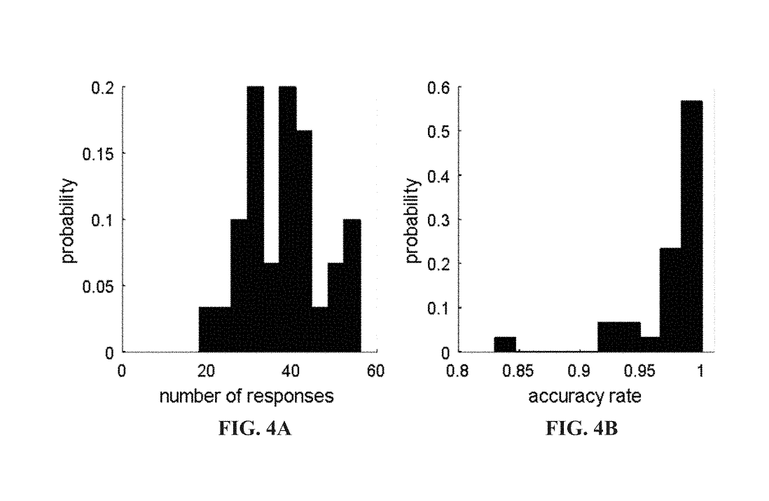

Smart phones with a 5.1 inch screen were programmed with suites for performing the eSDMT test. Test persons were asked to carry out the tests on the smart phone according to the instructions shown on the display. 30 subjects were investigated. The determined responses and accuracies are shown in FIGS. 4A-4B.

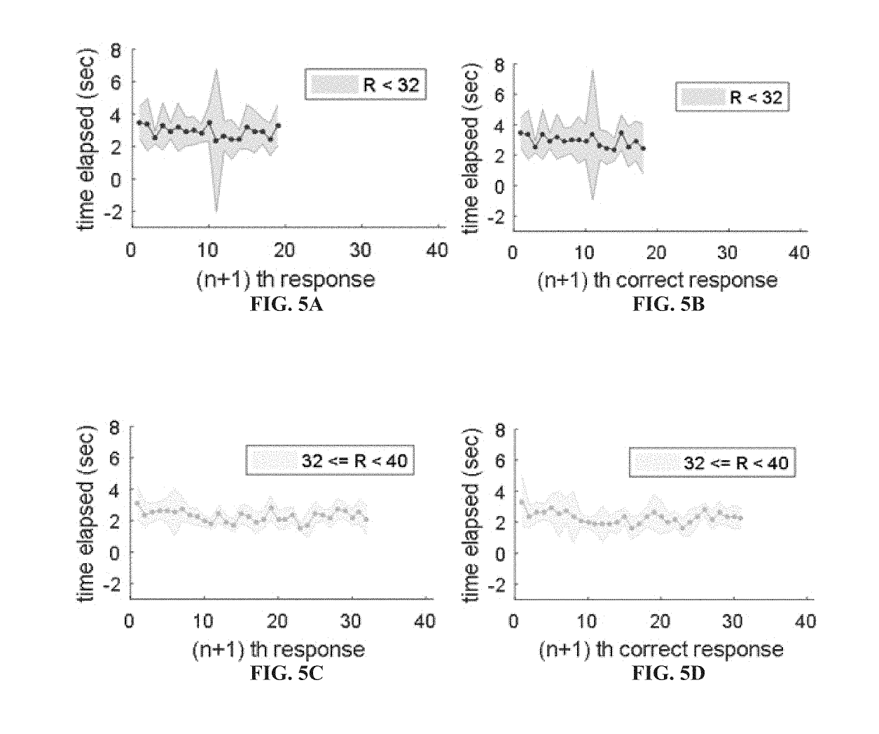

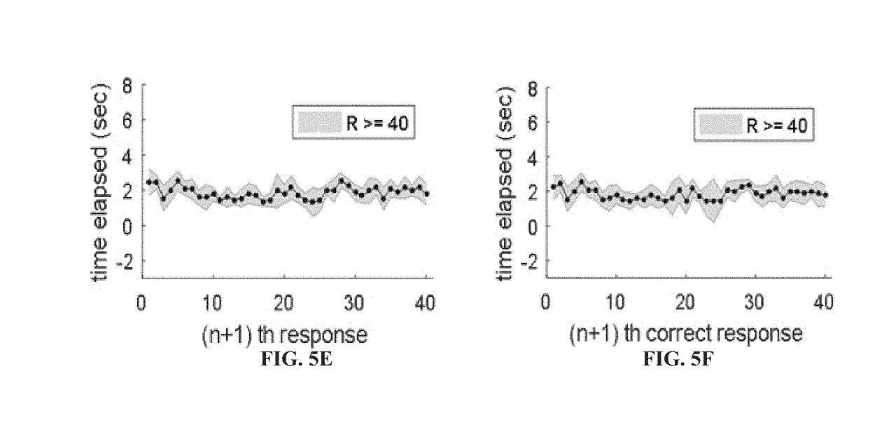

The time elapsed between subsequent responses (R) and subsequent correct responses (CR) was also investigated in the implemented eSDMT tests. Results are shown in FIGS. 5A-5F.

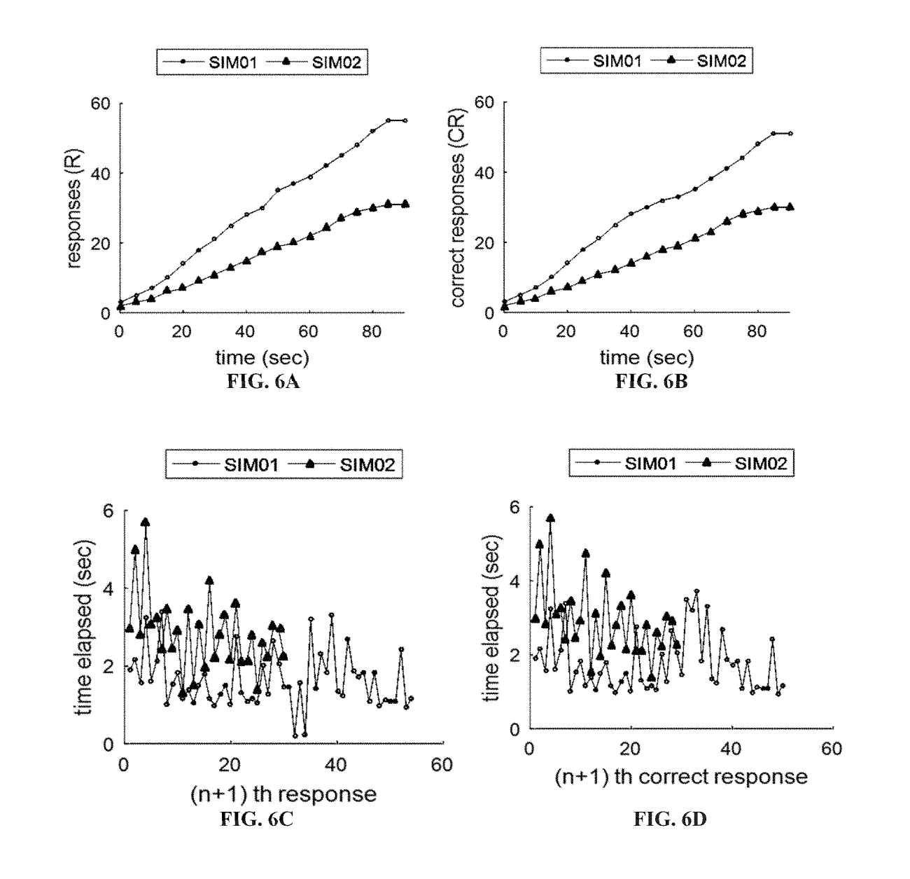

Furthermore, responses (R) and correct responses (CR) profiles were determined. Examples of responses (R) and correct responses (CR) profile of two subjects with quite distinct performances in eSDMT tests are shown in FIGS. 6A-6D.

### EXAMPLE 2

### A Computer-implemented Test Evaluating Fine Motoric Capabilities (Fine Motoric Assessments), in Particular, Hand Motor Functions and, in Particular, the Touchscreen-Based “Draw a Shape” and “Squeeze a Shape” Tests

Smart phones with a 5.1 inch screen were programmed with suites for performing the “Draw a Shape” and “Squeeze a Shape” tests. Test persons were asked to carry out the tests on the smart phone according to the instructions shown on the display.

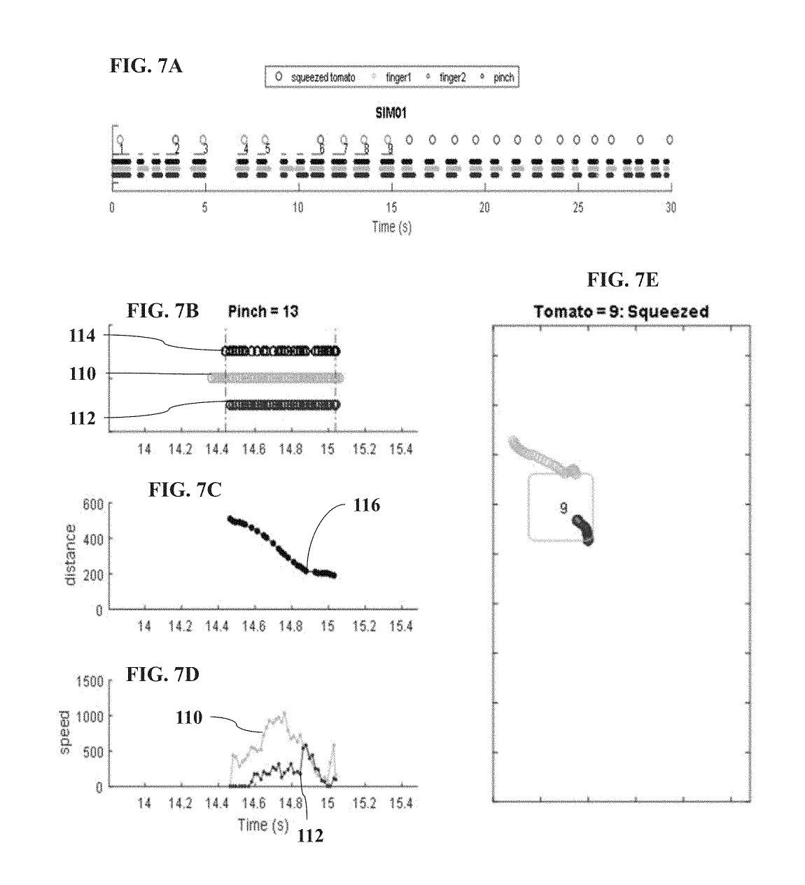

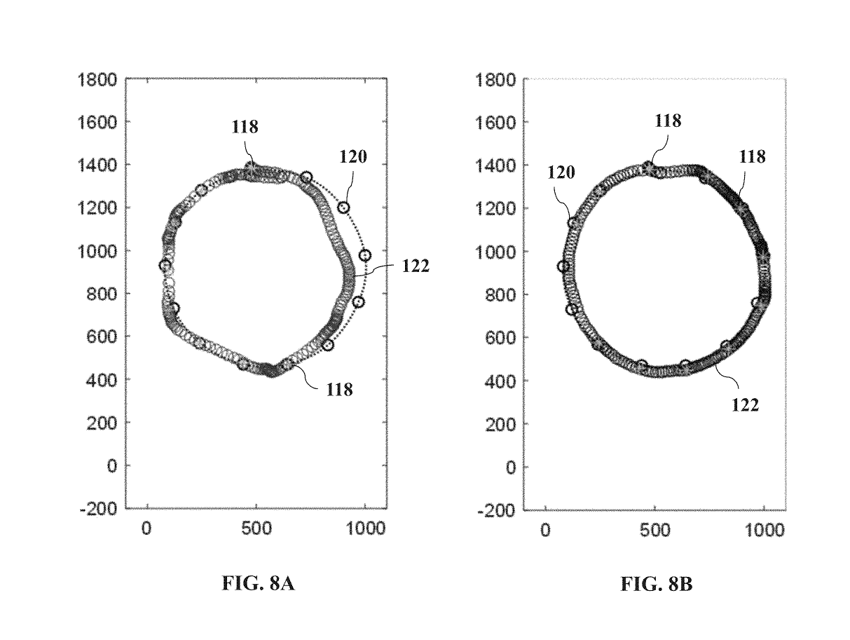

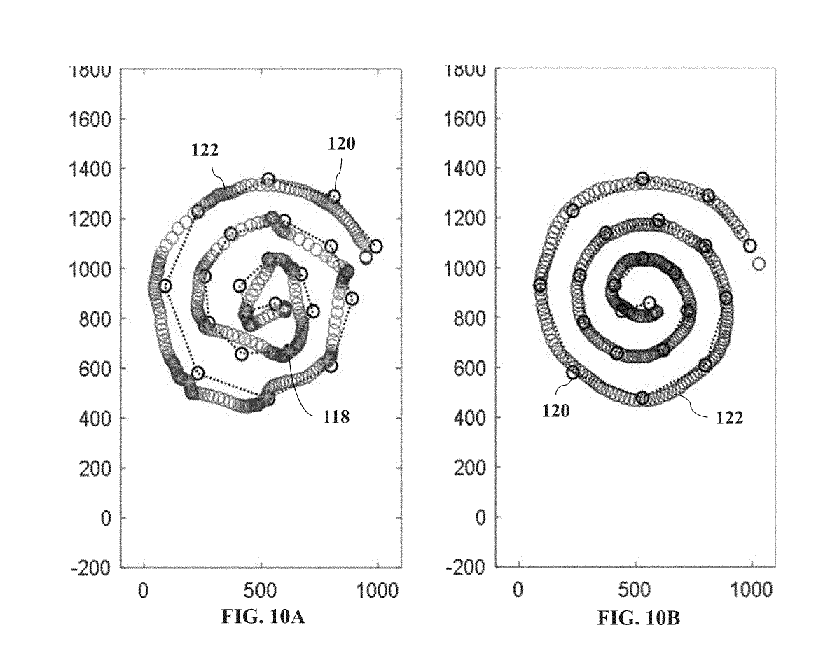

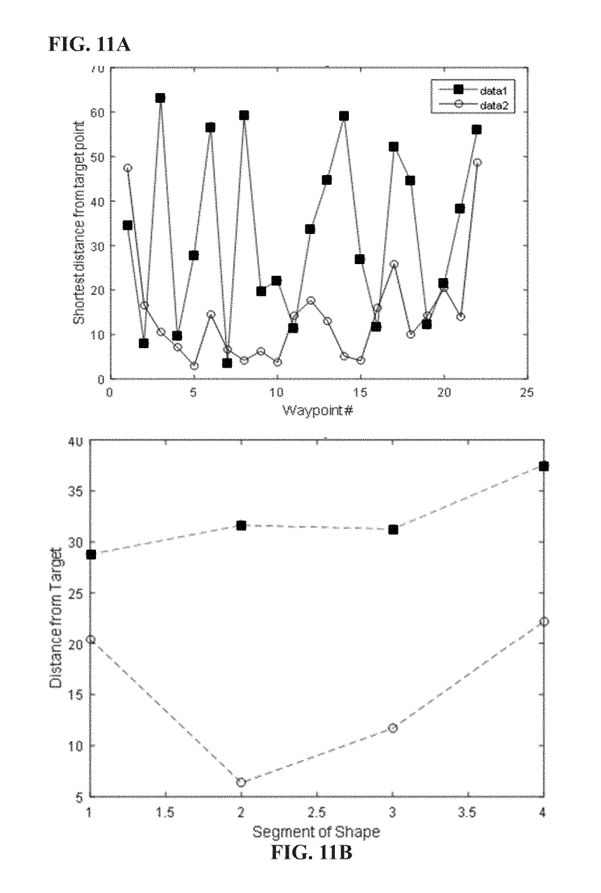

In the squeeze a shape set up, touch events from first and second fingers were determined and distances were calculated as well as the speed of the squeezing event (FIGS. 7A-7E). In the draw a shape set up, touch traces for the circle shapes were determined. Results are depicted in FIG. 8 or 10.

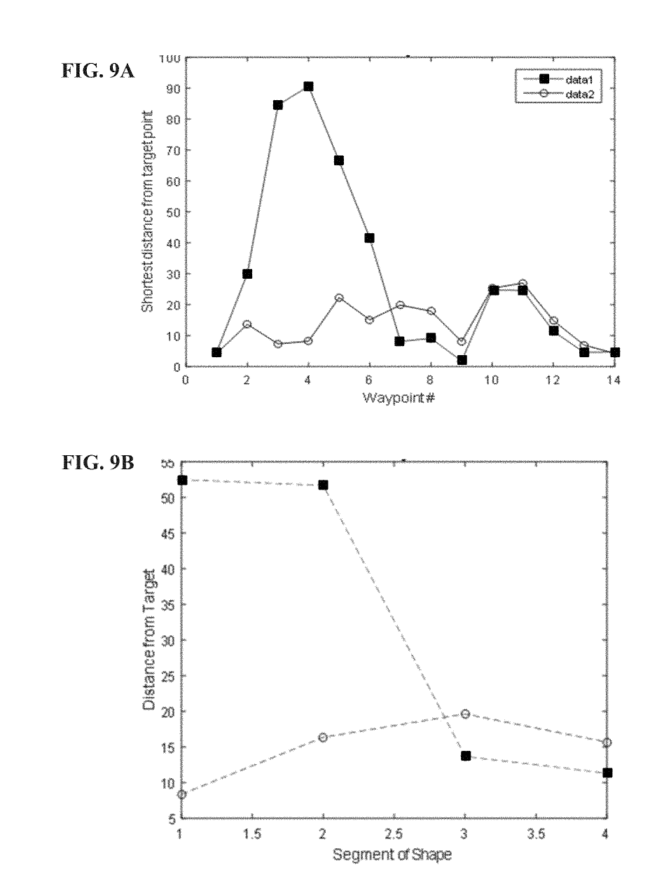

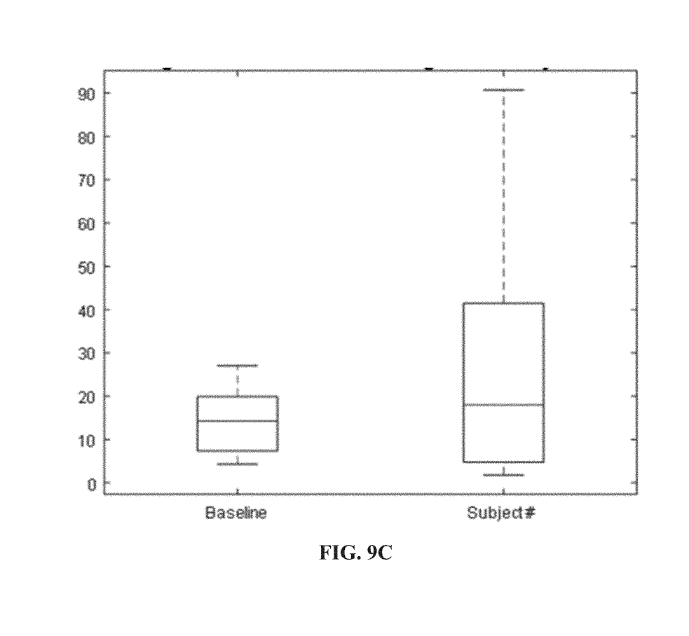

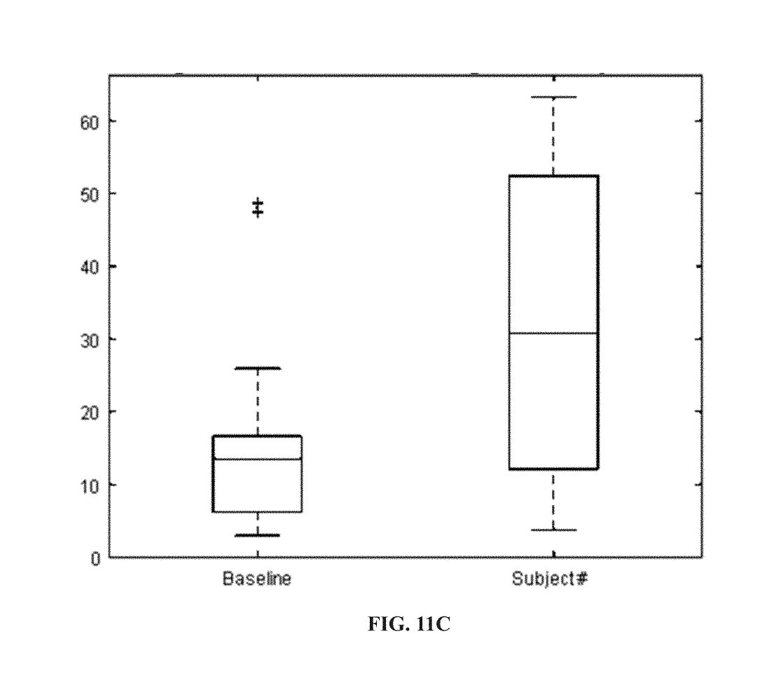

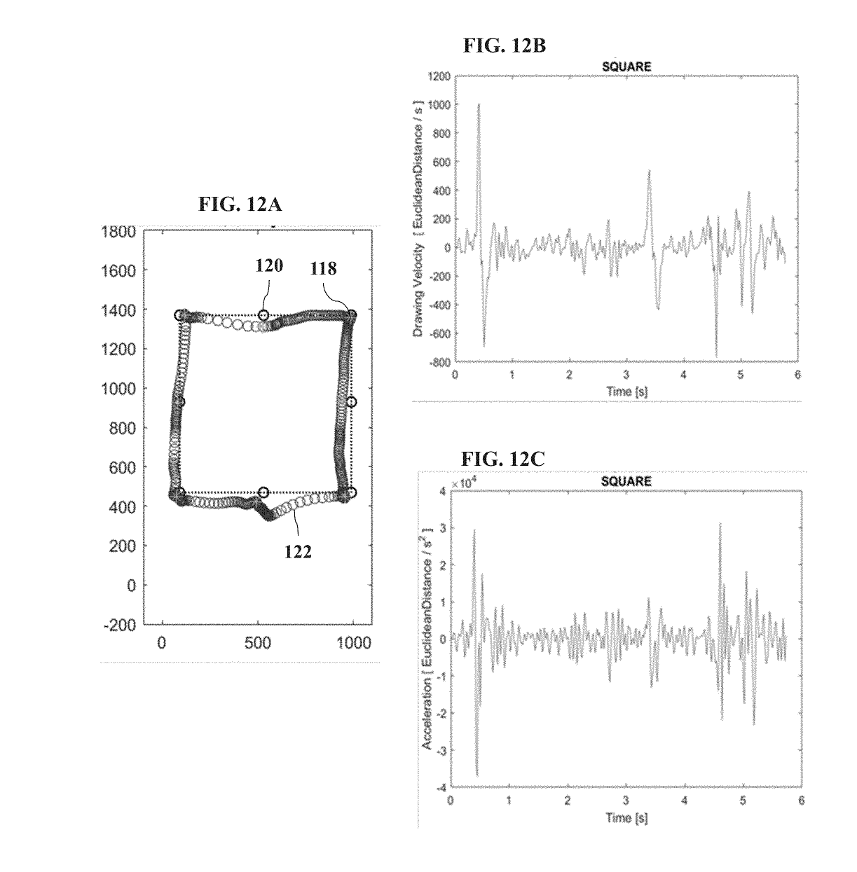

The overall calculated tracing performances are shown in FIGS. 9A-9C and FIGS. 11A-11C, respectively, and detailed data are summarized in Table 1 or 2, below.

TABLE 1
Circle assessment read-out performance statistics. The table lists
performance measures of the two traces depicted in FIGS. 8A-8B.

| | | | Time to | | | |
|---|---|---|---|---|---|---|
| | Number | Accu- | Complete | Total | Mean | Std. |
| | of Hits | racy | Shape [s] | Error | Error | Error |
| Baseline subject | 12 | 85.71% | 3.31 sec | 195.34 | 13.95 | 7.69 |
| Poor performing | 9 | 64.28% | 3.52 sec | 407.25 | 29.09 | 30.56 |
| subject | | | | | | |

TABLE 2
Spiral assessment read-out performance statistics. The table lists
performance measures of the two traces depicted in FIG. 10.

| | | | Time to | | | |
|---|---|---|---|---|---|---|
| | Number | Accu- | Complete | Total | Mean | Std. |
| | of Hits | racy | Shape [s] | Error | Error | Error |
| Baseline subject | 22 | 100% | 5.77 sec | 323.09 | 14.68 | 12.36 |
| Poor performing | 10 | 71.4% | 7.01 sec | 558.025 | 25.37 | 15.19 |
| subject | | | | | | |

Finally, spatial and temporal characteristics of a subject drawing a square were determined and results are shown in FIGS. 12A-12C.

### EXAMPLE 3

### Results from the Prospective Pilot Study (FLOODLIGHT) to Evaluate the Feasibility of Conducting Remote Patient Monitoring with the use of Digital Technology in Patients with Multiple Sclerosis

A study population will be selected by using the following inclusion and exclusion criteria:

Key inclusion criteria:
Signed informed consent form
Able to comply with the study protocol, in the investigator's judgment
Age 18-55 years, inclusive
Have a definite diagnosis of MS, confirmed as per the revised McDonald 2010 criteria
EDSS score of 0.0 to 5.5, inclusive
Weight: 45-110 kg
For women of childbearing potential: Agreement to use an acceptable birth control method during the study period

Key exclusion criteria:
Severely ill and unstable patients as per investigator's discretion
Change in dosing regimen or switch of disease modifying therapy (DMT) in the last 12 weeks prior to enrollment
Pregnant or lactating, or intending to become pregnant during the study

It is a primary objective of this study to show adherence to smartphone and smartwatch-based assessments quantified as compliance level (%) and to obtain feedback from patients and healthy controls on the smartphone and smartwatch schedule of assessments and the impact on their daily activities using a satisfaction questionnaire. Furthermore, additional objectives are addressed, in particular, the association between assessments conducted using the Floodlight Test and conventional MS clinical outcomes was determined, it was established if Floodlight measures can be used as a marker for disease activity/progression and are associated with changes in MRI and clinical outcomes over time and it was determined if the Floodlight Test Battery can differentiate between patients with and without MS, and between phenotypes in patients with MS.

In addition to the active tests and passive monitoring, the following assessments will be performed at each scheduled clinic visit:
Oral Version of SDMT
Fatigue Scale for Motor and Cognitive Functions (FSMC)
Timed 25-Foot Walk Test (T25-FW)
Berg Balance Scale (BBS)
9-Hole Peg Test (9HPT)
Patient Health Questionnaire (PHQ-9)
Patients with MS only:
Brain MRI (MSmetrix)
Expanded Disability Status Scale (EDSS)
Patient Determined Disease Steps (PDDS)
Pen and paper version of MSIS-29

While performing in-clinic tests, patients and healthy controls will be asked to carry/wear smartphone and smartwatch to collect sensor data along with in-clinic measures.

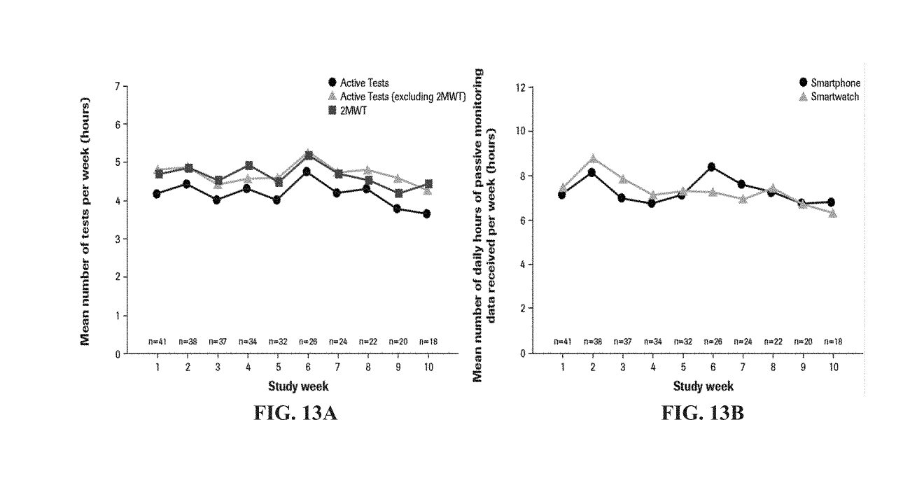

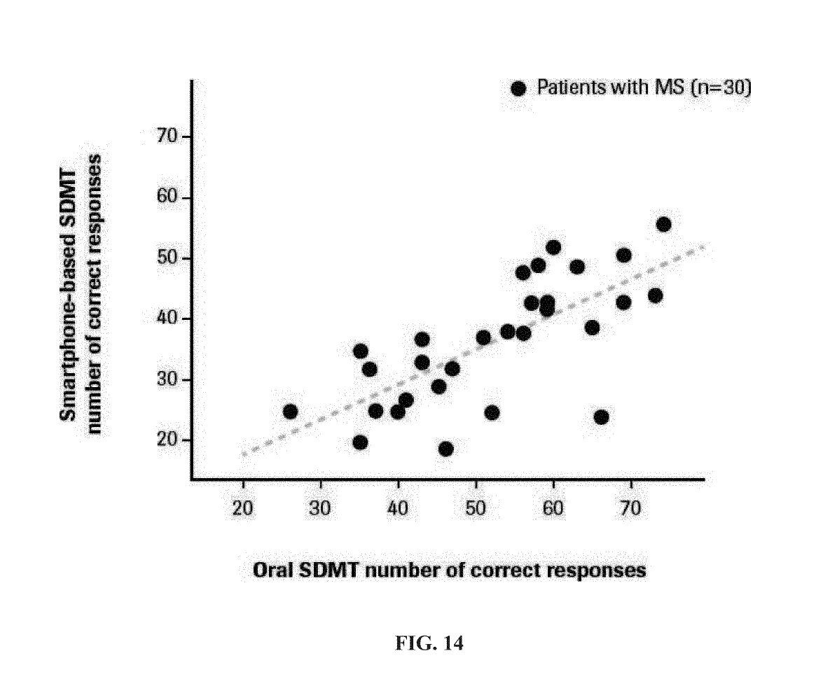

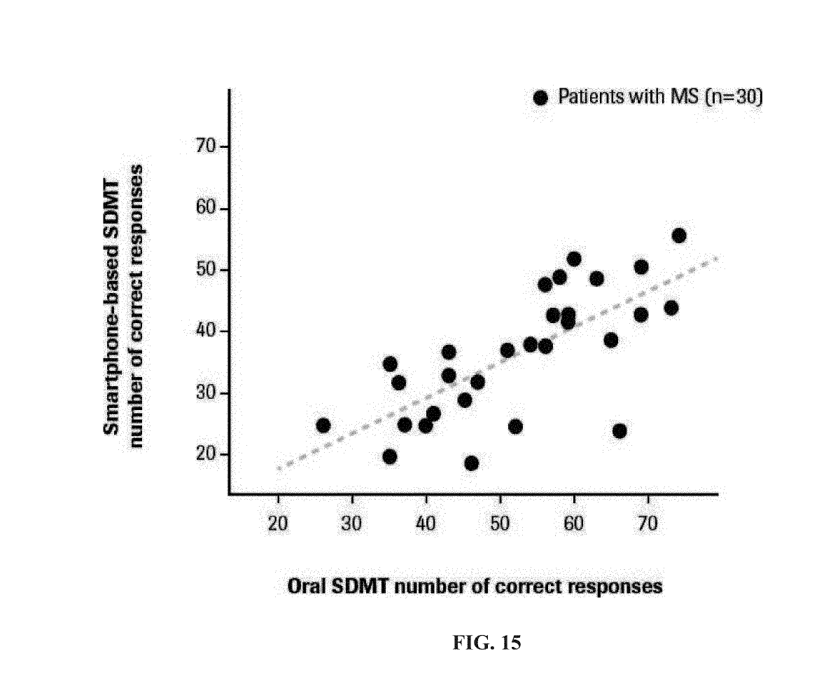

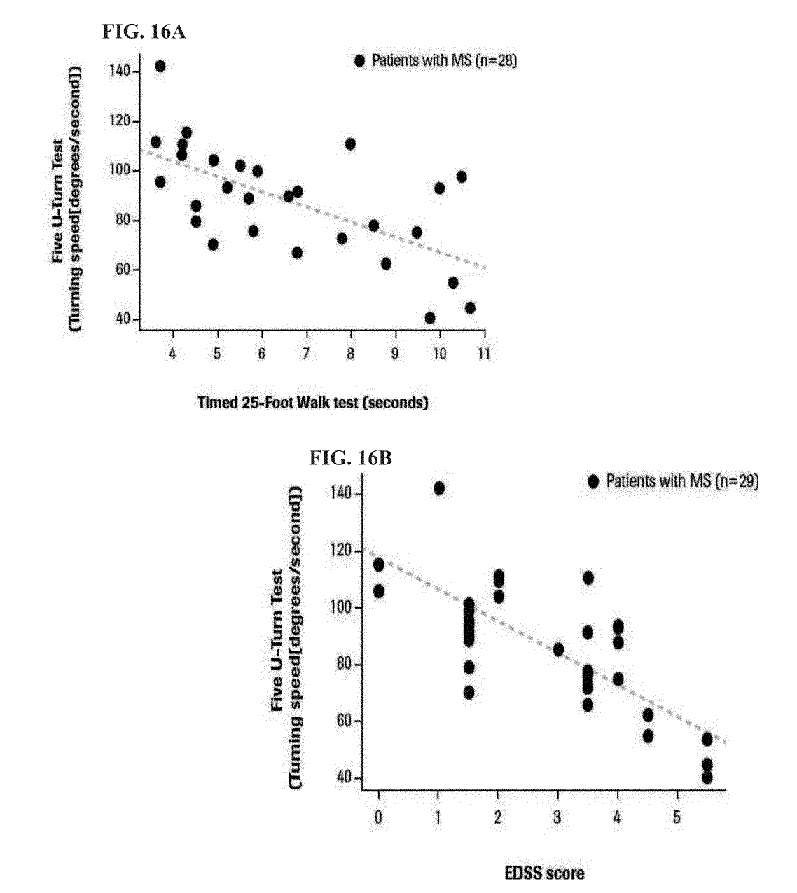

Patient adherence to active and passive testing is shown in FIGS. 13A-13B. Furthermore, the association between PROs performed in the hospital and on a mobile device (smart phone) are shown in FIG. 14. A baseline correlation was found between oral SDMT and mobile device implemented eSDMT was found; see FIG. 15. The turning speed while walking correlates with T25FW and EDSS; see FIGS. 16A-16B.

In summary, these results show that patients are highly engaged with the smartphone- and smartwatch-based assessments. Moreover, there is a correlation between tests and in-clinic clinical outcome measures recorded at baseline which suggests that the smartphone-based Floodlight Test Battery shall become a powerful tool to continuously monitor MS in a real-world scenario. Further, the smartphone-based measurement of turning speed while walking and performing U-turns appeared to correlate with T25FW and EDSS.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### CITED LITERATURE

Aktas 2005, Neuron 46, 421-432
Zamvil 2003, Neuron 38:685-688
Crawford A, et al. J Immunol 2006; 176(6):3498-506
Bar-Or A, et al. Ann Neurol 2010; 67(4):452-61
Lisak R P, et al. J Neuroimmunol 2012; 246(1-2):85-95
Weber M S, et al. Biochim Biophys Acta 2011; 1812(2):239-45
Serafini B, et al. Brain Pathol 2004; 14(2):164-74
Magliozzi R, et al. Ann Neurol 2010; 68(4):477-93
Bove 2015, Neurol Neuroimmunol Neuroinflamm 2 (6):e162
Link 2006, J Neuroimmunol. 180 (1-2): 17-28
Tsang 2011, Australian family physician 40 (12): 948-55
Compston 2008, Lancet 372(9648): 1502-17
Johnston 2012, Drugs 72 (9): 1195-211
Donnan 2008, Lancet. 371 (9624): 1612-23
Harbison 1999, Lancet. 353 (9168): 1935
Kidwell 1998, Prehospital Emergency Care. 2 (4): 267-73
Nor 2005, Lancet Neurology. 4 (11): 727-34
Burns 2009, The BMJ. 338: b158
Pasquier 1999, Journal of Neurology 246 (1):6-15
Jankovic 2008, Journal of Neurology, Neurosurgery, and Psychiatry. 79 (4): 368-376
Dayalu 2015, Neurologic Clinics. 33 (1): 101-14
Frank 2014, The journal of the American Society for Experimental NeuroTherapeutics. 11 (1): 153-60
Rao 2009, Gait Posture. 29 (3): 433-6
Zarei 2015, Surgical Neurology International. 6: 171
Polman 2011, Ann Neurol 69:292-302
Lublin 2014, Neurology 83: 278-286
Burns 2011, Neurology 76.7 Supplement 2: S6-S13
Waldemar 2007, European Journal of Neurology 14.1: e1-e26
Bäckman 2004, Journal of internal medicine 256.3: 195-204
Robert 2005, European Psychiatry 20.7: 490-496
Todd 2013, International journal of geriatric psychiatry 28.11: 1109-1124
Cheon 2002, Radiographics 22.3: 461-476
Gunstad 2006, Journal of Geriatric Psychiatry and Neurology 19.2: 59-64
Walker 2007, The Lancet 369.9557: 218-228
Strawn 2007, American Journal of Psychiatry 164.6: 870-876
Alsheikh 2015, arXiv preprint arXiv:1511.04664
Ordóñez 2016, Sensors, 16(1), 115
Hobart 2001, Brain 124: 962-73

What is claimed is:

1. A method for assessing a cognition and movement disease or disorder in a subject suspected to suffer therefrom, the method comprising:
a) determining at least one cognition or fine motoric activity parameter from a dataset of fine motoric activity measurements obtained from said subject using a mobile device;
b) comparing the determined at least one cognition or fine motoric activity parameter to a reference; and
c) assessing the cognition and movement disease or disorder based on the comparison.

2. The method of claim 1, wherein said cognition and movement disease or disorder is a disease or disorder of the central or peripheral nervous system affecting the pyramidal, extrapyramidal, sensory or cerebellar system, or a neuromuscular disease or is a muscular disease or disorder.

3. The method of claim 1, wherein said cognition and movement disease or disorder is selected from the group consisting of: multiple sclerosis (MS), neuromyelitis optica (NMO) and NMO spectrum disorders, stroke, a cerebellar disorder, cerebellar ataxia, spastic paraplegia, essential tremor, myasthenia and myasthenic syndromes or other forms of neuromuscular disorders, muscular dystrophy, myositis or other muscular disorders, a peripheral neuropathy, cerebral palsy, extrapyramidal syndromes, Parkinson's disease, Huntington's disease, Alzheimer's disease, other forms of dementia, leukodystrophies, autism spectrum disorders, attention-deficit disorders (ADD/ADHD), intellectual disabilities as defined by DSM-5, impairment of cognitive performances and reserve related to aging, Parkinson's disease, Huntington's disease, a polyneuropathy, motor neuron diseases and amyotrophic lateral sclerosis (ALS).

4. The method of claim 1, wherein step A0 comprises determining the at least one fine motoric activity parameter and the at least one fine motoric activity parameter is indicative for hand motoric functions.

5. The method of claim 1, wherein the dataset comprises data from a test encompassing drawing shapes with a finger or squeezing shapes with a finger on a sensor surface of the mobile device.

6. The method of claim 1, wherein the said dataset of cognition activity measurements comprises data from a test encompassing performing a eSDMT test on a sensor surface of the mobile device.

7. The method of claim 1, further comprising determining at least one performance parameter from a dataset of activity measurements that is indicative for the subject's other motoric capabilities and function, walking, color vision, attention, dexterity or cognitive capabilities, quality of life, fatigue, mental state, mood, vision or cognition.

8. The method of claim 1, further comprising determining at least one performance parameter from a dataset of activity measurements selected from the group consisting of: 2-Minute Walking Test (2MWT), 5 U-Turn Test (5UTT), Static balance test (SBT), Continuous Analysis of Gait (CAG), visual contrast acuity tests (such as low contrast letter acuity or Ishihara test), Mood Scale Question (MSQ), MSIS-29, and passive monitoring of all or a predetermined subset of activities of a subject performed during a certain time window.

9. The method of claim 1, wherein said mobile device has been adapted for carrying out on the subject one or more of the tests referred to in claim 4.

10. The method of claim 1, wherein said reference is at least one cognition or fine motoric activity parameter derived from a dataset of cognition or fine motoric activity measurements obtained from the subject at a time prior to the time when the dataset of cognition or fine motoric activity measurements referred to in step a) has been obtained from the subject.

11. The method of claim 10, wherein a worsening between the determined at least one cognition or fine motoric activity parameter and the reference is indicative for a subject that suffers from the cognition and movement disease or disorder.

12. The method of claim 1, wherein said reference is at least one cognition or fine motoric activity parameter derived from a dataset of cognition or fine motoric activity measurements obtained from a subject or group of subjects known to suffer from the cognition and movement disease or disorder, or wherein a determined at least one cognition or fine motoric activity parameter being essentially identical compared to the reference is indicative for a subject that suffers from the cognition and movement disease or disorder.

13. The method of claim 1, wherein said reference is at least one cognition or fine motoric activity parameter derived from a dataset of cognition or fine motoric activity measurements obtained from a subject or group of subjects known not to suffer from the cognition and movement disease or disorder.

14. The method of claim 13, wherein a determined at least one cognition or fine motoric activity parameter being worsened compared to the reference is indicative for a subject that suffers from the cognition and movement disease or disorder.

15. The method of claim 1 for use for recommending a therapy for a cognition and movement disease or disorder comprising the further step of recommending the therapy when the cognition and movement disease or disorder is assessed.

16. The method of claim 15, further comprising administering the therapy to a patient.

17. The method of claim 15, wherein the therapy comprises one or more of the following: drug-based therapies, surgery, psychotherapy, physical therapy, life-style recommendations, rehabilitation measures, nutritional diets.

18. The method of claim 15 wherein the therapy includes a drug-based therapy comprising one or more of: Interferon beta-1a, Interferon beta-1b, Glatiramer acetate, Mitoxantrone, Natalizumab, Fingolimod, Teriflunomide, Dimethyl fumarate, Alemtuzumab, Daclizumab, Thrombolytic agents, Acetylcholinesterase inhibitors, NMDA receptor antagonists, non-steroidal anti-inflammatory drugs, dopa carboxylase inhibitors, dopamine antagonists, MAO-B inhibitors, Amantadine, Anticholinergics, Tetrabenazine, Neuroleptics, Benzodiazepines, Riluzole.

19. The method of claim 1 for use in determining efficacy of a therapy against a cognition and movement disease or disorder comprising the further step of determining a therapy response if improvement of the cognition and movement disease or disorder occurs in the subject upon therapy or determining a failure of response if worsening of the cognition and movement disease or disorder occurs in the subject upon therapy or if the cognition and movement disease or disorder remains unchanged.

20. The method of claim 1, comprising carrying out steps a)-c) at least two times during a predefined monitoring period and determining whether the cognition and movement disease or disorder improves, worsens or remains unchanged in a subject.

21. A mobile device comprising a processor, at least one sensor, a database and software which is tangibly embedded in said device and, when running on said device, carries out the method of claim 1.

22. The mobile device of claim 21 for use in identifying a subject suffering from a cognition and movement disease or disorder, or for use in monitoring a subject suffering from a cognition and movement disease or disorder, in particular, in a real life, daily situation and on large scale, for investigating drug efficacy, e.g., also during clinical trials, in a subject suffering from a cognition and movement disease or disorder, for facilitating or aiding therapeutic decision making for a subject suffering from a cognition and movement disease or disorder, for supporting hospital management, rehabilitation measure management, health insurances assessments and management or supporting decisions in public health management with respect to subjects suffering from a cognition and movement disease or disorder or for supporting a subject suffering from a cognition and movement disease or disorder with life style or therapy recommendations.

23. A system comprising a mobile device comprising at least one sensor and a remote device comprising a processor and a database as well as software which is tangibly embedded in said device and, when running on said device, carries out the method of claim 1, wherein said mobile device and said remote device are operatively linked to each other.

24. The system of claim 23 for use in identifying a subject suffering from a cognition and movement disease or disorder, or for use in monitoring a subject suffering from a cognition and movement disease or disorder, in particular, in a real life, daily situation and on large scale, for investigating drug efficacy, e.g., also during clinical trials, in a subject suffering from a cognition and movement disease or disorder, for facilitating or aiding therapeutic decision making for a subject suffering from a cognition and movement disease or disorder, for supporting hospital management, rehabilitation measure management, health insurances assessments and management or supporting decisions in public health management with respect to subjects suffering from a cognition and movement disease or disorder or for supporting a subject suffering from a cognition and movement disease or disorder with life style or therapy recommendations.

25. A method for assessing a cognition and movement disease or disorder in a subject suspected to suffer therefrom, the method comprising:
a) prompting a subject to perform fine motoric activities using an electronic;
b) collecting with the electronic device a dataset of said fine motoric activities;
c) determining at least one cognition or fine motoric activity parameter from the dataset;
d) comparing the determined at least one cognition or fine motoric activity parameter to a reference; and
e) assessing the cognition and movement disease or disorder based on the comparison.

26. The method of claim 25 for use for recommending a therapy for a cognition and movement disease or disorder comprising the further step of recommending the therapy when the cognition and movement disease or disorder is assessed.

27. The method of claim 26, further comprising administering the therapy to a patient.

28. The method of claim 26, wherein the therapy comprises one or more of the following: drug-based therapies, surgery, psychotherapy, physical therapy, life-style recommendations, rehabilitation measures, nutritional diets.

29. The method of claim 26 wherein the therapy includes a drug-based therapy comprising one or more of: Interferon beta-1a, Interferon beta-1b, Glatiramer acetate, Mitoxantrone, Natalizumab, Fingolimod, Teriflunomide, Dimethyl fumarate, Alemtuzumab, Daclizumab, Thrombolytic agents, Acetylcholinesterase inhibitors, NMDA receptor antagonists, non-steroidal anti-inflammatory drugs, dopa carboxylase inhibitors, dopamine antagonists, MAO-B inhibitors, Amantadine, Anticholinergics, Tetrabenazine, Neuroleptics, Benzodiazepines, Riluzole.
